(19) United States
(12) Patent Application Publication
(10) Pub. No.: US 2016/0047826 A1
(43) Pub. Date: Feb. 18, 2016

(54) COMPOSITIONS AND METHODS FOR DETECTION OF A TARGET IN A MOLECULAR ASSAY USING PH CHANGES

(71) Applicant: McMaster University, Hamilton (CA)

(72) Inventors: Yingfu Li, Dundas (CA); Kha Tram, Hamilton (CA)

(73) Assignee: MCMASTER UNIVERSITY, Hamilton (CA)

(21) Appl. No.: 14/829,173

(22) Filed: Aug. 18, 2015

Related U.S. Application Data
(60) Provisional application No. 62/038,408, filed on Aug. 18, 2014.

(51) Int. Cl. G01N 33/84

(52) U.S. Cl. CPC G01N 33/84; G01N 2333/245; G01N 2333/33; G01N 2333/37; G01N 2333/186; G01N 2333/98

(57) ABSTRACT

The disclosure provides a sensor for detecting a target comprising a probe that is able to recognize the presence of the target; a pH-changing enzyme conjugated to an oligonucleotide that senses the recognition of the presence of the target by the probe; and a solid support linked or linkable to the probe; wherein the presence of the target causes the sensor to be either captured to the solid support or released into solution for detection of pH changes. Also provided are methods for using the sensor and kits.

### RELATED APPLICATIONS

This application claims benefit under 35 U.S.C. §119(e) to U.S. Provisional Patent Application Ser. No. 62/038,408 (pending), filed Aug. 18, 2014, incorporated herein by reference in its entirety.

### INCORPORATION OF SEQUENCE LISTING

A computer readable form of the Sequence Listing “3244-P46934US01_SequenceListing.txt” (8,192 bytes), submitted via EFS-WEB and created on Aug. 18, 2015, is herein incorporated by reference.

### FIELD

The disclosure relates to methods for rapid and simple detection of targets in a molecular assay. In particular, the disclosure relates to detection of pH changes in the presence of the target and subsequent colourimetric detection.

### BACKGROUND

Portable sensors are highly desirable for environmental monitoring, food safety control, and medical surveillance, particularly in resource-limited regions.¹⁻³ Colorimetric sensors represent an attractive option as the change of color can be easily detected by naked eyes. Litmus test for pH is a well-established and cheap colorimetric sensor that is still being widely used today. Existing litmus dyes and pH papers respond to pH change by producing a color signal.

Urease catalyzes the hydrolysis of urea into carbon dioxide and ammonia.⁴⁻⁶ The hydrolytic reaction raises the pH of the solution. Urease is highly efficient as it can speed up the hydrolysis of urea by ˜10¹⁴ times. Urease is also a stable enzyme and various forms of ureases are commercially available.^{7,8}

Functional nucleic acids, particularly DNA aptamers and aptazymes (aptamer-regulated DNAzymes), have been shown to be excellent molecular recognition elements because they offer high affinity and specificity for their cognate targets, and they are stable and cost-effective.⁹⁻¹⁸ Many aptazymes have been engineered using RNA-cleaving DNAzymes where target binding triggers the cleavage of an RNA-containing substrate.¹⁸

The ease of separation makes magnetic beads (MB) an attractive option to immobilize biomacromolecules,¹⁹ and thus they have been widely used to set up bioassays.²⁰⁻²³

### SUMMARY

The present inventors have devised sensors and methods that link a molecular recognition event to a pH change of the sensing solution, and takes advantage of inexpensive litmus dyes and pH papers for detection of targets. In particular, the present inventors have coupled a molecular recognition event to the activity of urease.

Herein the present inventors have demonstrated a simple and inexpensive litmus test for bacterial detection. The method takes advantage of a bacteria-specific RNA-cleaving DNAzyme probe as the molecular recognition element and the ability of urease to hydrolyze urea and elevate the pH of the test solution. By coupling urease to the DNAzyme on magnetic beads, the detection of bacteria is translated into a pH increase, which can be readily detected using a litmus dye or pH paper. The simplicity, low cost and broad adaptability make this litmus test attractive for field applications, particularly in the developing world.

The present inventors have extended the linkage of molecular recognition events to pH changes to other molecular detections, including detection of uranium, fungus, RCA amplification, viruses, ATP and other bacteria.

Accordingly, the present disclosure provides a sensor for detecting a target comprising:

a) a probe that is able to recognize the presence of the target;

b) a pH-changing enzyme conjugated to an oligonucleotide that senses the recognition of the presence of the target by the probe; and

c) a solid support linked or linkable to the probe;

wherein the presence of the target causes the sensor to be either captured to the solid support or released into solution for detection of pH changes.

In an embodiment, the pH changes are detectable by a pH meter, dye or paper, or colourimetric dye or paper, such as litmus paper.

In an embodiment, the pH changing enzyme is urease.

In another embodiment, the solid support is a magnetic bead, glass, plastic or paper.

In yet another embodiment, the target is any compound that is able to be detected by a probe, such as a DNA, an RNA, a protein, a small molecule, a cell, a chemical compound or an ion.

In an embodiment, the probe is any molecule that is able to recognize the presence of the target, such as a DNA, RNA, DNAzyme, ribozyme, an aptamer, an amplified DNA product, or an aptazyme.

In one embodiment, the probe is an RNA cleaving DNAzyme with an RNA linkage that cleaves the RNA linkage in the presence of target, and wherein the oligonucleotide conjugated to the pH-changing enzyme is complementary to a portion of the RNA-containing sequence that when cleaved by the DNAzyme is no longer linked or capable of linking to the solid support. The RNA cleaving DNAzyme, in an embodiment, cleaves the RNA in the presence of the target: uranyl ions or E. coli bacteria.

In another embodiment, the probe is a biotinylated primer capable of acting as a forward primer (or a reverse primer) to amplify a portion of the target and wherein the oligonucleotide conjugated to the pH-changing enzyme is complementary to a portion of the reverse primer (or a forward primer) to amplify a portion of the target. In an embodiment, the target is C. difficile. In such an embodiment, forward primers may be used to distinguish between different strains of C. difficile such as use of the forward primers comprising one or more of SEQ ID NOs:1-3 and the reverse primer comprising SEQ ID NO:4. In an alternate embodiment, the target is a fungus, such as Pythium aphanidermatum.

In yet another embodiment, the probe is a nicked DNA having a first end and a second end and the oligonucleotide conjugated to the pH-changing enzyme is complementary to a portion of the sequence overlapping the second end and wherein the target is ATP which is required for a ligation reaction to occur.

In a further embodiment, the probe is a biotinylated primer and a circular DNA, wherein the primer is capable of amplifying the circular DNA by rolling circle amplification and the oligonucleotide conjugated to the pH-changing enzyme is complementary to a portion of the amplified DNA, wherein the target is the amplified DNA.

In yet a further embodiment, the probe is a biotinylated oligonucleotide complementary to a portion of a target DNA or RNA and the oligonucleotide conjugated to the pH-changing enzyme is complementary to a different portion of the target DNA or RNA. In an embodiment, the target is viral DNA, such as hepatitis C viral DNA.

Also provided herein is a method of detecting a target in solution comprising:

a) incubating a sensor described herein with the target in solution to allow the probe to recognize the target and the pH-changing enzyme-oligonucleotide conjugate to sense the recognition;

b) (i) removing the solution from the solid support after incubation if the pH-changing enzyme is releasable upon recognition of the target by the probe; or (ii) washing the solid support after incubation if the enzyme is capturable upon recognition of the target by the probe to produce a solution containing the washed solid support;

c) incubating the solution of b) i) or ii) with a substrate of the enzyme;

d) testing the pH of the solution of c)
wherein a change in pH is indicative of the presence and/or quantity of the target in the initial solution.

In one embodiment, the pH is tested using litmus paper or dyes. In another embodiment, the pH is tested using a pH paper or meter.

In an embodiment, wherein the pH changing enzyme is urease and substrate is urea.

In another embodiment, the solid support is a magnetic bead. The magnetic bead may be directly linked to the probe. Alternatively, the magnetic bead may be linkable to the probe, for example, the magnetic bead may be conjugated to streptavidin and the probe may be biotinylated such that the probe is linkable to the magnetic bead by the streptavidin-biotin interaction. Accordingly in one embodiment, the linking of the probe to the solid support is after the probe recognizes the target and the pH-changing enzyme-oligonucleotide conjugate senses the recognition but before b).

In an embodiment, the target is any compound that is recognizable by the probe, such as a DNA, an RNA, a protein, a small molecule, a cell, a chemical compound or an ion.

In another embodiment, the probe is any molecule that recognizes the target, such as a DNA, RNA, DNAzyme, ribozyme, an aptamer, an amplified DNA product, or an aptazyme.

In one embodiment, the probe is an RNA cleaving DNAzyme that cleaves the RNA in the presence of target, and wherein the oligonucleotide conjugated to the pH-changing enzyme is complementary to a portion of the RNA-containing sequence that when cleaved by the DNAzyme is no longer linked to the solid support and is released into solution after a); wherein in b) i) the solution is removed and wherein in c) the substrate is added to the removed solution and wherein in d) the pH of the removed solution is tested. In an embodiment, the RNA cleaving DNAzyme cleaves the RNA in the presence of target, such as uranyl ions or E. coli bacteria.

In another embodiment, the probe is a biotinylated primer capable of acting as a forward primer (or reverse primer) to amplify a portion of target and the oligonucleotide conjugated to the pH-changing enzyme is complementary to a portion of the reverse primer (or forward primer) to amplify a portion of the target, such that the amplified product is linked to the solid support and the urease is attached to the end of the amplified product after a), wherein the solid support is then washed in b) ii) and then wherein in c) substrate is added to the washed solid support in solution and wherein in d) the pH of the solid support in solution is tested. In an embodiment, the target is C. difficile and primers are used to detect C. difficile strains, such as forward primers comprising SEQ ID NOs:1-3 and reverse primer comprising SEQ ID NO:4. In an alternate embodiment, the target is a fungus, such as Pythium aphanidermatum.

In yet another embodiment, the probe is a nicked DNA having a first end and a second end and wherein the oligonucleotide conjugated to the pH-changing enzyme is complementary to a portion of the sequence overlapping the second end and wherein the target is ATP which is required for a ligation reaction to occur; such that in the presence of ATP the ligation reaction occurs and the pH-changing enzyme binds to the ligated DNA, which is linked to the solid support after a), wherein the solid support is then washed in b) ii) and then wherein in c) substrate is added to the washed solid support in solution and wherein in d) the pH of the solid support in solution is tested.

In a further embodiment, the probe is a biotinylated primer and a circular DNA, wherein the primer is linkable to a solid support and is capable of amplifying the circular DNA by rolling circle amplification and wherein the oligonucleotide conjugated to the pH-changing enzyme is complementary to a portion of the amplified DNA, wherein the target is the amplified DNA and the pH-changing enzyme binds to the amplified DNA which is linked to the solid support after a), wherein the solid support is then washed in b) ii) and then wherein in c) substrate is added to the washed solid support in solution and wherein in d) the pH of the solid support in solution is tested.

In yet a further embodiment, the probe is a biotinylated oligonucleotide complementary to a portion of a target DNA or RNA and the oligonucleotide conjugated to the pH-changing enzyme is complementary to a different portion of the target DNA or RNA; such that in the presence of the target, the pH-changing enzyme is captured and linked to the solid support after a), wherein the solid support is then washed in b) ii) and then wherein in c) substrate is added to the washed solid support in solution and wherein in d) the pH of the solid support in solution is tested. In an embodiment, the target is viral DNA, such as HCV DNA.

The disclosure further provides kits comprising the sensors described herein for practicing the methods described herein.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows conceptual schematics of an embodiment. (A) Cleavage reaction. The binding of the cognate target to the aptazyme on the magnetic bead triggers its cleavage activity, resulting in the release of urease. (B) Colorimetric reporting assay. Upon target-induced cleavage and magnetic separation, the urease is taken to hydrolyze urea in the presence of a litmus dye.

FIG. 2 shows the synthesis and a functional test of a sensor construct. (A) Conjugation of 5′-amino modified DNA to urease using MBS. (B) Analysis of DNA-urease conjugation mixtures using non-denaturing PAGE. (C) Functional test. Clv: cleavage product; Unclv: uncleaved construct. Note: EC1 was radioactively labeled.

FIG. 3 shows the litmus test with reaction buffer alone (left tube of each panel), CCE prepared from 10⁷ of B. subtilis cells (middle), or E. coli cells (right). The photographs were taken at 0, 15, 30 and 60 minutes.

FIG. 4 shows monitoring pH changes caused by the presence of E. coli via electronic reading with a hand-held pH meter (A) and pH paper strips (B).

FIG. 5 shows a Litmus test with CCE-EC prepared from varying numbers of E. coli cells. The photograph was taken after a signal-producing time of 1 hour (top panel) or 2 hours (bottom).

FIG. 6 shows color responses of six pH-sensitive dyes in the hydrolytic reaction of urea by urease. MB-EC1-UrDNA was treated with reaction buffer (RB) only, CCE-BS prepared from 10⁷ Bacillus subtilis cells, or CCE-EC from 10⁷ E. coli cells for 2 hours. After magnetic separation, the supernatant from each cleavage reaction (RB, CCE-BS and CCE-EC) was incubated with a urea solution containing one of the following 6 dyes: 1, bromothymol blue; 2, phenol red; 3, neutral red; 4, cresol red; 5, m-cresol purple; 6, o-cresolphthalein complexone. For each dye, the left, middle and right tubes contained RB only, CCE-BS and CCE-EC, respectively.

FIG. 7 shows detection of a single colony-forming unit of E. coli using the litmus test following varying hours of culturing.

FIG. 8 shows the general concept for the detection of Uranyl ions.

FIG. 9 shows (A) Litmus test for 0.015 mg/L of Uranyl in water, well-water, and lake water. (B) Colour change visualized using pH paper strips at 0 and 15 minutes in the presence and absence of 0.015 mg/L of Uranyl.

FIG. 10 shows a schematic illustration of a colourimetric test after PCR.

FIG. 11 shows sequence design and location of tcdC target. Three forward primers were designed to target different sites of the tcdC gene for differentiating specific strains of C. difficile (forward primers: SEQ ID NOs:1-3, respectively and reverse primer: SEQ ID NO:4).

FIG. 12 shows detection of tcdC variants. In the absence of C. difficile (NC) no amplified products were produced resulting in no colour change. The presence of C. difficile with different tcdC variants generated a specific amplification pattern, which can be observed through 2% agarose gel electrophoresis (A). Successful amplification of DNA molecules captured the Ur-DNA and generated an observable colour change (B).

FIG. 13 shows sensitivity of C. difficile detection using PCR. Serial dilutions of C. difficile cells were used to determine the sensitivity of the assay. After PCR, a clear visual band was detected with 2×10⁵ number of cells (A). When the litmus test was performed, after 60 minutes a slight colour change was observed at 2×10³ number of cells.

FIG. 14 shows a litmus test for P. aphanidermatum. A sensitivity test was performed to detect P. aphanidermatum after 35 cycles of PCR. A visible colour change after 1 hr from yellow to red was observed at 20 attomolar (aM).

FIG. 15 shows a schematic diagram of ATP detection using colourimetric detection.

FIG. 16 shows ligation of S1 and S2 in the presence of ATP. Ligation reactions were prepared in the presence (+) or absence (−) of ATP. The products of these reactions were loaded onto a 10% denaturing PAGE gel using Urea as a denaturant. A ladder (L) containing oligonucleotides of length 90, 80, 50, and 40 nt, and marker (M) containing oligonucleotides of length 88, 46, and 42 nt were also loaded. The gels were stained with 1×SYBR Gold fluorescent dye in Tris-borate-EDTA buffer for 30 min at 40° C., and fluorescence was detected at 200 microns using the Typhoon 9410 Multipurpose Scanner from GE Healthcare. The 88 nt, 46 nt, and 42 nt bands corresponded to the expected ligation product (Lig), S2, and S1 respectively.

FIG. 17 shows specificity of the colourimetric ATP test. A test sample containing ATP was compared to samples containing potential interfering molecules as shown, and a negative control (NC) containing no target for colour change. All tests shown contained 100 nM of their respective molecule. Samples were allowed to incubate for 15 minutes after addition of urea solution, and images were captured at 0 and 15 minutes using a Canon PowerShot G11 camera. Only in the presence of ATP will there be a clear change of colour from yellow to red.

FIG. 18 shows sensitivity of the colourimetric ATP test. The colourimetric assay was tested for distinguishable colour change at various concentrations of ATP. The concentrations tested were (from left to right), as well as a negative control containing no ATP (NC). Samples were allowed to incubate for 15 minutes after addition of urea solution, and images were captured at 0 and 15 minutes using a Canon PowerShot G11 camera. After 15 minutes, a clear colour change can be observed with an ATP concentration of 10 nM.

FIG. 19 shows a schematic illustration of the RCA litmus system.

FIG. 20 shows the colourimetric test comparing the unamplified method (A) versus RCA (B). Amplified RCA products captured more Ur-DNA in solution. Higher concentrations of captured Ur-DNA facilitated urea hydrolysis resulting in faster rate of colour change. The RCA method allowed for more than 100-fold signal enhancement.

FIG. 21 shows a schematic illustration of oligonucleotide detection.

FIG. 22 shows detection of HCV DNA. In the absence of HCV DNA (NC), Ur-DNA was not able to hybridize with the 5′-biotin DNA probe and resulted in no colour change. Conversely, the presence of HCV DNA rapidly generates a colour change from yellow to red and after 15 minutes, a visible colour change was observed to as low as 0.13 nM of HCV DNA.

### DETAILED DESCRIPTION

The present inventors have demonstrated that a molecular recognition event of a target may be coupled to a pH-changing enzyme, such that colourimetric detection of the target is possible.

Accordingly, the present disclosure provides a sensor for detecting a target comprising:

a) a probe that is able to recognize the presence of the target;

b) a pH-changing enzyme conjugated to an oligonucleotide that senses the recognition of the presence of the target by the probe; and

c) a solid support linked or linkable to the probe;

wherein the presence of the target causes the sensor to be either captured to the solid support or released into solution for detection of pH changes.

In an embodiment, the pH changes are detectable by a pH meter, dye or paper, or colourimetric dye or paper, such as litmus paper.

In an embodiment, the pH changing enzyme is urease. Urease in the presence of its substrate urea produces ammonia which increases the pH of the solution.

The probe may be linked or linkable to any solid support. The solid support can be any solid support that is capable of linking or being linkable to the probe, such as a magnetic bead, glass, plastic and paper. The phrase “linked to the solid support” refers to when the probe is actually attached to the solid support. The phrase “linkable to the solid support” refers to a probe that has the ability to link to the solid support, for example, where a probe is biotinylated and the solid support has streptavidin on it, the probe has the ability to link to the solid support through the formation of the streptavidin-biotin complex.

In an embodiment, the solid support is a magnetic bead such that magnetization can facilitate the separation of the solid support and its attachments or linkages from the remaining solution so that the remaining solution can be removed easily. Alternative solid supports that can also facilitate the separation of the solid support and its attachments or linkages from the remaining solution are materials such as glass, plastic, and paper that can be chemically functionalized to be linked or linkable to the probe.

The target may be any compound that is able to be detected by a probe. For example, the target can be a DNA, an RNA, a protein, a small molecule, a cell, a chemical compound or an ion.

The probe may be any molecule that is able to recognize the presence of the target or a compound that triggers a molecular recognition event. For example, the probe can be a DNA, RNA, DNAzyme, a ribozyme, an aptamer, an amplified DNA product, or an aptazyme. A DNA or RNA probe or an amplified product may recognize a target by the presence of a sequence that is complementary to the target to allow for Watson-Crick base pairing. A DNAzyme, ribozyme or aptazyme may recognize a target that would then allow for cleavage of the RNA substrate specific to the DNAzyme, ribozyme or aptazyme triggering the recognition event. An aptamer may initiate the recognition event through the formation of secondary and/or tertiary structures to accommodate binding of the target through non-covalent interactions.

The present inventors have demonstrated that use of an RNA cleaving DNAzyme that cleaves the RNA in the presence of E. coli or Uranyl ions, can be sensed by an oligonucleotide conjugated urease that is complementary to a portion of the RNA containing sequence such that when cleavage occurs, the urease-oligonucleotide is released into solution and the pH of the solution is indicative of the amount of cleavage or the amount of target.

Accordingly, in one embodiment, the probe is an RNA cleaving DNAzyme having an RNA linkage that cleaves the RNA linkage in the presence of target, and wherein the oligonucleotide conjugated to the pH-changing enzyme is complementary to a portion of the RNA-containing sequence that when cleaved by the DNAzyme is no longer linked or capable of linking to the solid support.

An RNA-cleaving DNAzyme has two components: the enzyme and substrate (the RNA portion). In one embodiment, both components are linked in one long sequence (see the E. coli example). In another embodiment, the two components exist as two separate strands (see the uranyl example). If it is one long sequence, the DNAzyme contains the biotin. If it is two separate sequences, the RNA substrate will contain the biotin and be linkable to the solid support and the DNAzyme part of the probe hybridizes to the substrate rather than links to the solid support.

DNAzymes and their targets are known in the art and may be used in the sensors and methods disclosed herein. For example, RNA cleaving DNAzyme with an RNA linkage that cleaves the RNA linkage in the presence of breast cancer, lead ions, manganese ions, magnesium ions, and zinc ions.⁵⁹⁻⁶³

Accordingly, the RNA cleaving DNAzyme, in an embodiment, cleaves the RNA in the presence of the target: uranyl ions or E. coli bacteria.

The present inventors have also demonstrated that the use of an oligonucleotide conjugated to urease complementary to a reverse primer is able to detect an amplified product produced from PCR with various forward primers that have been biotinylated, specific to different C. difficile strains. In this example, the oligonucleotide conjugated urease is captured by the solid support through the interaction of the biotin. The present inventors further demonstrated that the same concept can be used to detect the presence of the fungus Pythium aphanidermatum.

Accordingly, in another embodiment, the probe is a biotinylated primer capable of acting as a forward primer (or reverse primer) to amplify a portion of the target and wherein the oligonucleotide conjugated to the pH-changing enzyme is complementary to a portion of the reverse primer (or forward primer) to amplify a portion of the target. In an embodiment, the target is C. difficile. In such an embodiment, forward primers may be used to distinguish between different strains of C. difficile such as use of the forward primers comprising one or more of SEQ ID NOs:1-3 and the reverse primer comprising SEQ ID NO:4. In an alternate embodiment, the target is a fungus, such as Pythium aphanidermatum.

PCR can be used to identify any target species given access to their genetic material and amplification of genetic targets can be used in the sensors and methods disclosed herein. For example, any bacteria known to cause infectious diseases or food-borne pathogens such as Salmonella, and Listeria monocytogenes can be isolated and their genetic material used for amplification.

The present inventors have further demonstrated the use of an oligonucleotide conjugated to urease for the sensing of ATP in a solution by taking advantage of the need for ATP in a ligation reaction. In such a reaction, the urease is captured when the ligation reaction occurs and the oligonucleotide conjugated to urease binds the ligated product.

Accordingly, in yet another embodiment, the probe is a nicked DNA having a first end and a second end and the oligonucleotide conjugated to the pH-changing enzyme is complementary to a portion of the sequence overlapping the second end and wherein the target is ATP which is required for a ligation reaction to occur.

The present inventors have further shown that an oligonucleotide conjugated to urease is also useful in the detection of the amount of amplified DNA produced in a rolling circle amplification. In such a reaction, the oligonucleotide conjugated to urease is complementary to a portion of the amplified product and thus the urease is bound to the DNA repeats and the pH changes in the solution caused by the hydrolysis of urea by the oligonucleotide conjugated urease allows for determination of the amount of amplification.

Accordingly, in a further embodiment, the probe is a biotinylated primer and a circular DNA, wherein the primer is capable of amplifying the circular DNA by rolling circle amplification and the oligonucleotide conjugated to the pH-changing enzyme is complementary to a portion of the amplified DNA, wherein the target is the amplified DNA.

The present inventors have also shown that simple Watson-Crick pairing of an oligonucleotide (conjugated to urease) to a target DNA or RNA is able to allow for detection of the target DNA or RNA when a second biotinylated probe also binds a portion of the target.

Accordingly, in yet a further embodiment, the probe is a biotinylated oligonucleotide complementary to a portion of a target DNA or RNA and the oligonucleotide conjugated to the pH-changing enzyme is complementary to a different portion of the target DNA or RNA. In an embodiment, the target is viral DNA, such as hepatitis C viral DNA.

Demonstration of viral DNA is one example of using short DNA or RNA sequences as targets for the use in the sensors and methods disclosed herein. For example, microRNA found in plants, animals, and some viruses may also be targets in the sensors and methods disclosed herein.

Also provided herein are methods for using the sensors disclosed herein.

Accordingly, herein provided is a method of detecting a target in solution comprising:

a) incubating a sensor described herein with the target in solution to allow the probe to recognize the target and the pH-changing enzyme-oligonucleotide conjugate to sense the recognition;

b) (i) removing the solution from the solid support after incubation if the pH-changing enzyme is releasable upon recognition of the target by the probe; or (ii) washing the solid support after incubation if the enzyme is capturable upon recognition of the target by the probe to produce a solution containing the washed solid support;

c) incubating the solution of b)i) or ii) with a substrate of the enzyme;

d) testing the pH of the solution of c)
wherein a change in pH is indicative of the presence and/or quantity of the target in the initial solution.

A person skilled in the art would readily know the conditions necessary for incubating a colorimetric sensor with the target in solution to allow the probe to recognize the target. Such conditions are known in the art and will depend on the molecular recognition event, i.e. probes and target used.

In one embodiment, the pH is tested using litmus paper or dyes. In another embodiment, the pH is tested using a pH paper or meter.

In an embodiment, the pH changing enzyme is urease and in c) the substrate is urea. In such an embodiment, urease catalyzes the conversion of urea to ammonia, which increases the pH of the solution, which can then be detected in step d).

The solid support can be any solid support that is capable of linking or being linkable to the probe, such as a magnetic bead, glass and plastic. The phrase “linked to the solid support” refers to when the probe is actually attached to the solid support. The phrase “linkable to the solid support” refers to a probe that has the ability to link to the solid support, for example, where a probe is biotinylated and the solid support has streptavidin on it, the probe has the ability to link to the solid support through the formation of the streptavidin-biotin complex.

In an embodiment, the solid support is a magnetic bead. The magnetic bead may be directly linked to the probe. Alternatively, the magnetic bead may be linkable to the probe, for example, the magnetic bead may be conjugated to streptavidin and the probe may be biotinylated such that the probe is linkable to the magnetic bead by the streptavidin-biotin interaction. In step b), the solid support can be segregated by applying magnetization to the container containing the solution to more easily allow for washing and/or separation of the solution.

In one embodiment, the linking of the probe to the solid support is after the probe recognizes the target and the pH-changing enzyme-oligonucleotide conjugate senses the recognition but before b).

In an embodiment, the target is any compound that is recognizable by the probe, such as a DNA, an RNA, a protein, a small molecule, a cell, a chemical compound or an ion.

In another embodiment, the probe is any molecule that recognizes the target, such as a DNA, RNA, DNAzyme, ribozyme, an aptamer, an amplified DNA product, or an aptazyme.

The washing solution in b) ii) can be any buffering solution that does not disrupt, denature or inactivate the probe or the pH-changing enzyme.

In one embodiment, the probe is an RNA cleaving DNAzyme having an RNA linkage that cleaves the RNA linkage in the presence of target, and wherein the oligonucleotide conjugated to the pH-changing enzyme is complementary to a portion of the RNA-containing sequence that when cleaved by the DNAzyme is no longer linked to the solid support and is released into solution after a); wherein in b) i) the solution is removed and then wherein in c) substrate is added to the removed solution and wherein in d) the pH of the removed solution is tested. In an embodiment, the RNA cleaving DNAzyme cleaves the RNA in the presence of target, such as uranyl ions or E. coli bacteria.

In another embodiment, the probe is a biotinylated primer capable of acting as a forward primer (or reverse primer) to amplify a portion of target and the oligonucleotide conjugated to the pH-changing enzyme is complementary to a portion of the reverse primer (or forward primer) to amplify a portion of the target, such that the amplified product is linked to the solid support and the urease is attached to the end of the amplified product after a), wherein the solid support is then washed in b) ii) and then wherein in c) substrate is added to the washed solid support in solution and wherein in d) the pH of the solid support in solution is tested. In an embodiment, the target is C. difficile and primers are used to detect C. difficile strains, such as forward primers comprising SEQ ID NOs:1-3 and reverse primer comprising SEQ ID NO:4. In an alternate embodiment, the target is a fungus, such as Pythium aphanidermatum.

In yet another embodiment, the probe is a nicked DNA having a first end and a second end and wherein the oligonucleotide conjugated to the pH-changing enzyme is complementary to a portion of the sequence overlapping the second end and wherein the target is ATP which is required for a ligation reaction to occur; such that in the presence of ATP the ligation reaction occurs and the pH-changing enzyme binds to the ligated DNA, which is linked to the solid support after a), wherein in b) ii) the solid support is then washed, wherein in c) the substrate is added and wherein in d) the pH of the solid support in solution is tested.

In a further embodiment, the probe is a biotinylated primer and a circular DNA, wherein the primer is linkable to a solid support and is capable of amplifying the circular DNA by rolling circle amplification and wherein the oligonucleotide conjugated to the pH-changing enzyme is complementary to a portion of the amplified DNA, wherein the target is the amplified DNA and the pH-changing enzyme binds to the amplified DNA which is linked to the solid support after a), wherein in b) ii) the solid support is then washed, wherein in c) the substrate is added and wherein in d) the pH of the solid support in solution is tested.

In yet a further embodiment, the probe is a biotinylated oligonucleotide complementary to a portion of a target DNA or RNA and the oligonucleotide conjugated to the pH-changing enzyme is complementary to a different portion of the target DNA or RNA; such that in the presence of the target, the pH-changing enzyme is captured and linked to the solid support after a), wherein in b) ii) the solid support is then washed, wherein in c) the substrate is added and wherein in d) the pH of the solid support in solution is tested. In an embodiment, the target is viral DNA, such as HCV DNA.

Also provided herein is a kit comprising a sensor disclosed herein. The kit may additionally comprise reagents necessary for carrying out the methods disclosed herein, including a wash solution, litmus paper or other pH detecting paper, and instructions for use.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Detection of E. coli

### Results

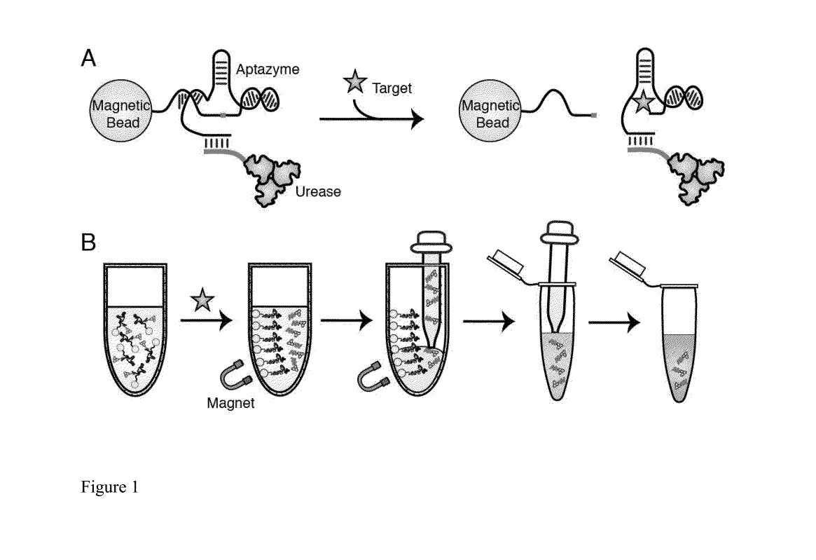

The conceptual framework is illustrated in FIG. 1. Four components are utilized: streptavidin-coated magnetic beads (MB), an aptazyme, urease conjugated to a DNA oligonucleotide (UrDNA) and a pH-sensitive dye (or pH paper). The aptazyme contains a biotin moiety at its 5′ end for streptavidin binding and a sequence extension at its 3′ end for hybridization with UrDNA. Thus, simple mixing of the MB, the aptazyme, and the UrDNA results in functional MB that can release urease in response to the target of the aptazyme (FIG. 1A). Upon magnetic separation, the freed urease can be taken to hydrolyze urea in the presence of a litmus dye for color generation (FIG. 1B).

The above design is compatible with any RNA-cleaving aptazyme; however for the current demonstration, a DNAzyme, EC1, previously developed for the specific detection of Escherichia coli (E. coli), a model bacterial pathogen was employed.^{24, 25} Pathogenic bacteria pose a grave threat to public health and safety, and early detection of specific pathogens is an important step towards preventing a potential outbreak. However, laborious and expensive pathogen tests often represent a bottleneck in such efforts, particularly in resource-limited regions. A simple litmus test for pathogen detection offers a very attractive option.

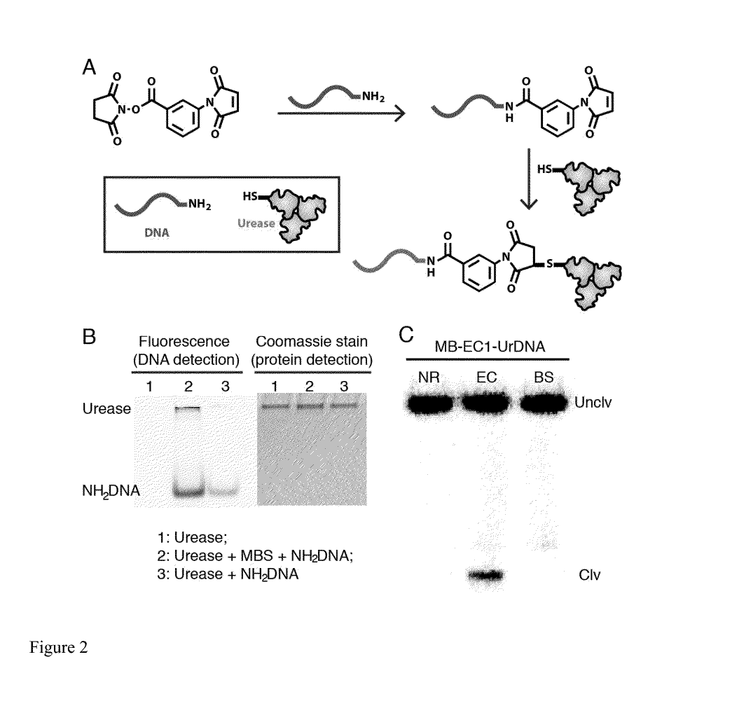

A bifunctional linker, maleimidobenzoic acid N-hydroxy-succinimide ester (MBS), was used to achieve the conjugation of a 5′-amino modified DNA oligonucleotide (H₂N-DNA) to urease (FIG. 2A). H₂N-DNA was first allowed to react with MBS, resulting in maleimidobenzoic DNA amide (MDA). This was followed by the coupling of urease to MDA via thiol addition to the double bond of the maleimide. Using a fluorescently labeled DNA, this method was able to achieve successful coupling of H₂N-DNA to urease (FIG. 2B).

The functionality of MB-EC1-UrDNA was examined by treating the MB conjugates with the crude cellular extract (CCE) prepared from E. coli (EC; intended bacteria) or Bacillus subtilis (BS; a negative control; it was previously shown that EC1 cannot be activated by CCEs from a host of bacteria including B. subtilis^{24,25}). The cleavage activity was analyzed by the denaturing polyacrylamide gel electrophoresis (dPAGE); for this reason, EC1 was internally labeled with ³²P so that the cleavage of EC1 would result in a DNA fragment that can be detected by dPAGE. MB-EC1-UrDNA was activated by CCE-EC but not by CCE-BS (FIG. 2C).

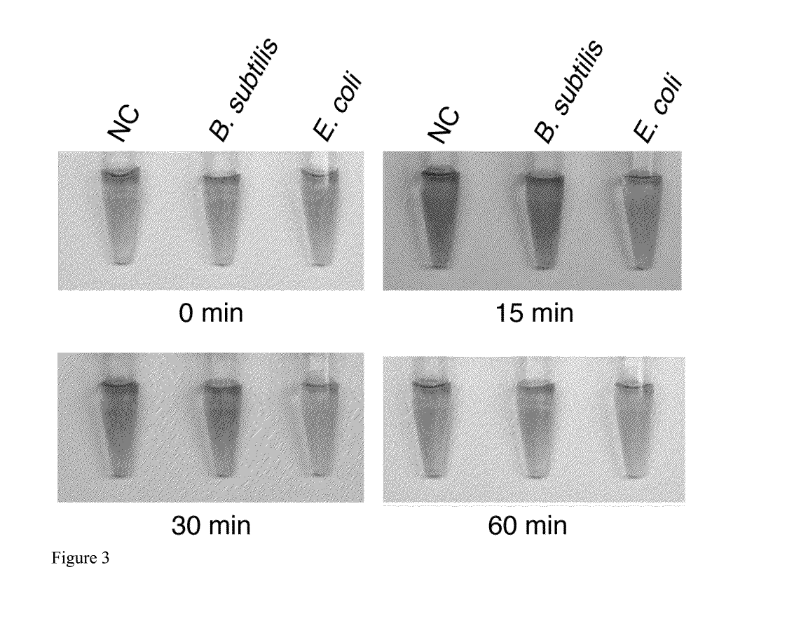

The litmus test for E. coli was next carried out using phenol red as the litmus dye because it produced a rather sharp, yellow-to-pink transition. The procedure consisted of two separate reactions: E. coli induced probe cleavage reaction and urease mediated reporting reaction. The cleavage reaction was conducted at room temperature for 60 minutes in 1× reaction buffer (1×RB; 10 mM HEPES, pH 7.4, 150 mM NaCl, 15 mM MgCl₂, 0.01% Tween 20) containing CCE-EC or CCE-BS prepared from 10⁷ E. coli or B. subtilis cells (total reaction volume was 10 μL). This was followed by 10-fold dilution with H₂O to facilitate the magnetic separation and minimize the impact of the buffering agent on the reporting reaction. Following magnetic separation, 70 μL of the diluted cleavage solution were mixed with 100 μL of urea-containing solution (2 M NaCl, 60 mM MgCl₂, 50 mM urea, 1 mM HCl) and 10 μL of 0.04% phenol red. This resulted in a new reaction mixture with an initial pH of −5.5; at this pH, phenol red exhibits a yellow color. As shown in FIG. 3, within 15 minutes, the reaction mixture from CCE-EC changed its color from yellow to brownish pink, which continued to intensify into bright pink within 60 minutes. In sharp contrast, the color of the reaction mixture originated either from RB alone or from CCE-BS remained unchanged.

Several other dyes were then examined for the same assay, which included bromothymol blue, neutral red, cresol red, m-cresol purple, and o-cresolphthalein complexone and the data is presented in FIG. 6. It is apparent from the results that any of these dyes are compatible with the assay.

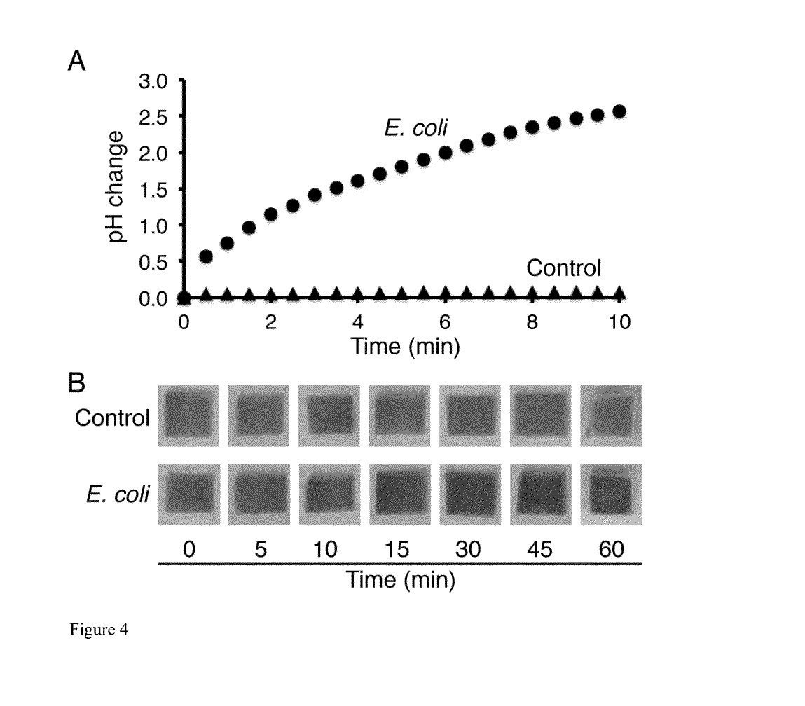

The time-dependent pH increase of the reporting solution was next measured using a hand-held pH meter and the data is shown in FIG. 4A. The pH increased nearly 3 pH units for the E. coli sample while the pH of the control samples (either buffer only or B. subtilis samples) remained unchanged.

The pH changes of these samples were also monitored using commercially available pH paper strips and the data is provided in FIG. 4B. Once again, while the control samples produced no detectable color change on the pH paper, a notable color change can be detected in 10 minutes with the E. coli sample. The results from all three experiments above show that the devised method can be used to achieve target-specific detection using simple methods that include color change of litmus dyes in solution, color change of a pH paper and electronic readings using a hand-held pH meter.

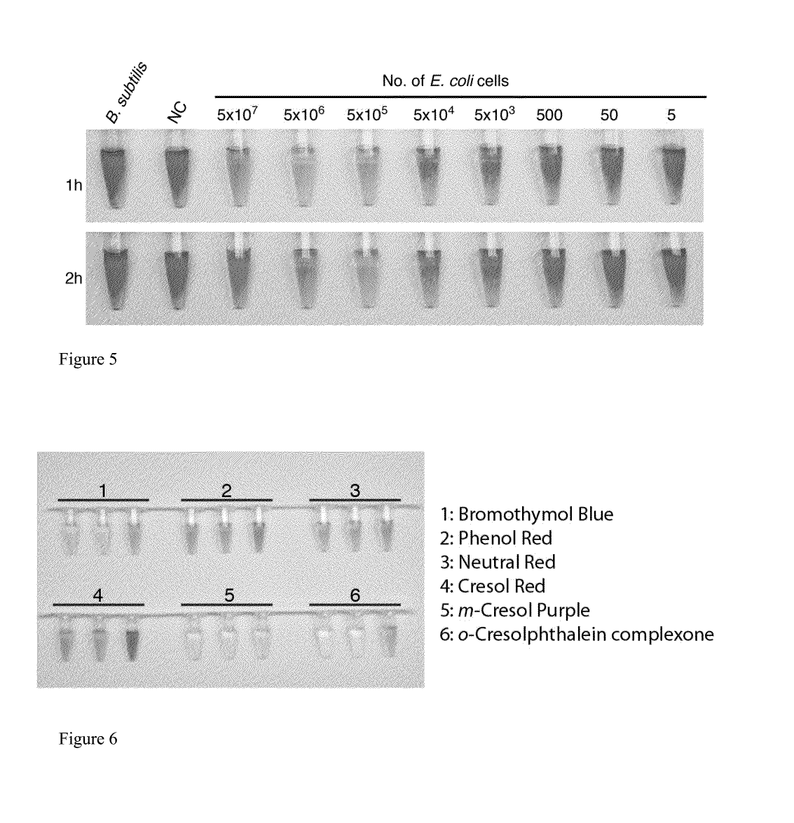

The sensitivity of the assay was determined using phenol red. Eight CCE-EC samples were prepared from serially diluted E. coli samples, each of which contained the specific number of cells given in FIG. 5. A sharp color transition was observed for the sample containing 5×10⁵ cells after color development for both 1 hour (top panel) or 2 hours (bottom panel). A subtle but detectable color transition, in comparison to the two reference samples (5×10⁷ B. subtilis cells and RB alone), was observed for the sample containing 5,000 cells for 1-hour incubation and 500 cells for the 2-hour incubation.

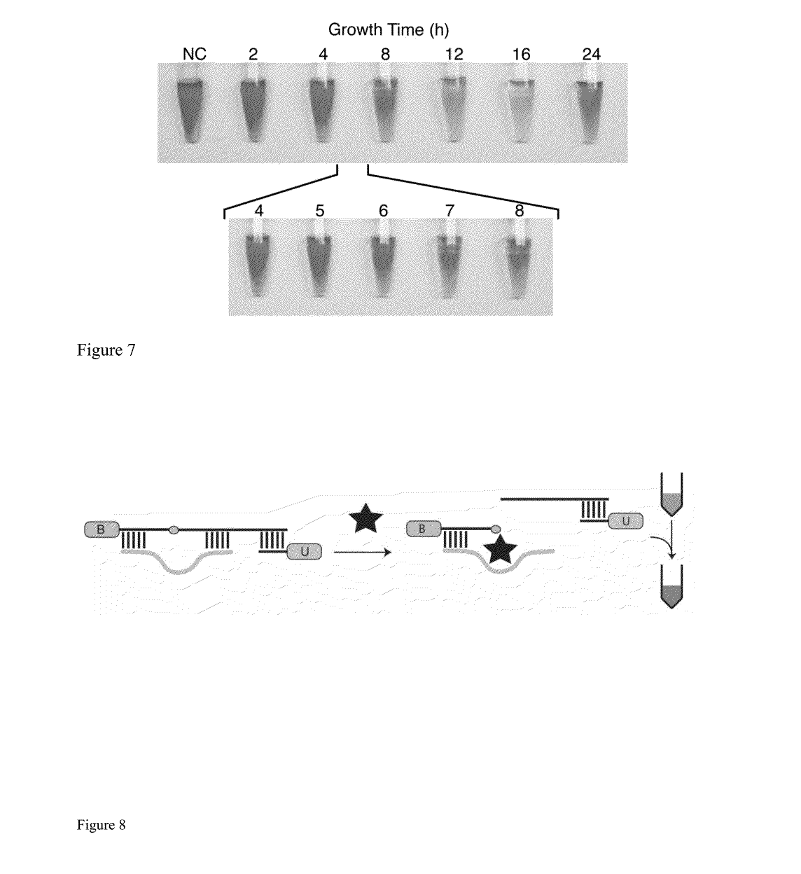

The capability of the litmus test for the detection of a single cell of E. coli was also examined (i.e., 1 colony forming unit, CFU) following a culturing step. As shown in FIG. 7, the combined culturing-litmus test can easily detect a single CFU as early as 7 hours of culturing.

In summary, a litmus test for E. coli was developed that uses an RNA-cleaving DNAzyme as the molecular recognition element and protein enzyme urease as the signal transducer. The sensing system also takes advantage of magnetic separation that is easy to implement and pH-sensitive dyes or pH paper strips that are cheap and widely available. The litmus test exhibits a sensitivity similar to that of fluorescence based detection method previously published using the same DNA probe, however the colorimetric test is simple to perform and does not require specialized equipment, and therefore is better suited for field applications, particularly in developing countries.

Although an E. coli sensing aptazyme was used in the current study, the sensor design can be easily extended to any RNA-cleaving aptazyme. Similarly, the design principle should be broadly compatible with any system in which a cleavable substrate (for the detection for an enzyme or factors that activate the enzyme) can be coupled to urease.

### Materials and Methods

Enzymes and Chemicals.

T4 DNA ligase, T4 polynucleotide kinase (PNK) and ATP were purchased from Thermo Scientific. [γ-³²P]dATP were purchased from Perkin Elmer. Streptavidin coated magnetic beads of 1.5 μm (BioMag-SA) was purchased from Bangs Laboratories Inc. Urease powder from Canavalia ensiformis (Jack bean), maleimidobenzoic acid N-hydroxy-succinimide ester (MBS), phenol red, bromothymol blue sodium salt, neutral red, cresol red, m-cresol purple, o-cresolphthalein complexone, were obtained from Sigma-Aldrich. All other chemicals were purchased from Bioshop Canada and used without further purification. The water used in this study was double-deionized (ddH₂O) and autoclaved.

Synthesis and Purification of Oligonucleotides.

Five synthetic oligonucleotides were used in this study; their sequences and functions are provided in Table 1. All these DNA oligonucleotides were purchased from Integrated DNA Technologies (IDT) and purified by 10% denaturing (8 M urea) polyacrylamide gel electrophoresis (dPAGE), and their concentrations were determined spectroscopically.

Synthesis of the Aptazyme EC1.

DE1 (2 nmol) was phosphorylated (reaction volume: 50 μL) at 37° C. with 10 units (U) of PNK in 1×PNK buffer A containing 2 mM ATP (final concentration) for 20 min. The reaction was quenched by heating the mixture at 90° C. for 5 min. Upon cooling to room temperature, equimolar BS1 and T1 were added. The resultant DNA mixture was then heated to 90° C. for 1 min and cooled to room temperature. Then, 10 μL of 10×DNA ligase buffer and 10 U of T4 DNA Ligase were added, along with enough ddH₂O to make the final volume to be 100 μL. This was followed by incubation at room temperature for 2 h. The DNA was then concentrated by ethanol precipitation and the ligated EC1 was purified by 10% dPAGE.

TABLE 1

| Name | Labels | Sequence | Note |
|---|---|---|---|
| BS1 | 5′-Biotin; adenine | TTTTT TTTTT TTACT | Substrate |
| SEQ ID | ribonucleotide (R) | CTTCC TAGCF RQGGT | |
| NO: 5 | | TCGAT CAAGA | |
| DE1 | None | GATGT GCGTT GTCGA | DNAzyme with an |
| SEQ ID | | GACCT GCGAC CGGAA | extension (italic |
| NO: 6 | | CACTA CACTG TGTGG | letters) that can |
| | | GGATG GATTT CTTTA | hybridize to LD1 |
| | | CAGTT GTGTG TTGAA | |
| | | CGCTG TGTCA AAAAA AAAA | |
| T1 | None | GACAA CGCAC ATCTC | Template for |
| SEQ ID | | TTGAT CGAAC C | ligating BS1 to |
| NO: 7 | | | DE1 |
| LD1 | 5′-NH2 | TTTTT TTTTT TTTTT | DNA for coupling |
| SEQ ID | | TGACA CAGCG TTCAA | to urease |
| NO: 8 | | | |
| LD2 | 5′-NH₂ and 3′- | TTTTT TTTTT TTTTT | FAM-tagged LD1 |
| SEQ ID | FAM | TGACA CAGCG TTCAA | |
| NO: 9 | | | |

For the labeling of the same construct with ³²P, 2 nmol of DE1 was phosphorylated (reaction volume: 50 μL) at 37° C. with 10 U of PNK in 1×PNK buffer A containing 10 μCi [γ-³²P]ATP for 20 min. This was followed by addition of 2 μL of 100 mM ATP and further incubation at 37° C. for 20 min. The rest of the procedure was identical to the one described above for the synthesis of non-radioactive EC1.

DNA-Urease Conjugation.

An MBS solution was made by dissolving 2 mg MBS in 1 mL of dimethyl sulphoxide (DMSO). Similarly a urease solution was produced by dissolving 1.5 mg urease powder in 1 mL of 1×PBS buffer (pH 7.2). 1 nmol LD1 (or LD2) and 3.2 μL of MBS solution were mixed and adjusted to a final reaction volume of 100 μL with 1×PBS buffer, and allowed to react at room temperature. After 2 h, the mixture was passed through a membrane based molecular sizing centrifugal column with a molecular weight cut-off of 3,000 Daltons (NANOSEP OMEGA, Pall Incorporation) to remove excess MBS. The column was washed with 50 μL of 1×PBS buffer 3 times and the DNA was resuspended in 100 μL of 1×PBS buffer. The urease solution (1 mL) was then added to the MBS activated DNA. The conjugation reaction was allowed to proceed at room temperature for 1 h. The mixture was filtered through 300,000-Dalton cut-off centrifugal column. The DNA-Urease conjugate (UrDNA) was then washed with 50 μL of 1×PBS buffer 3 times, and resuspended in 100 μL of 1×PBS buffer.

Probe Immobilization.

First, 100 μL of MB suspension was placed in a magnet holder to separate the supernatant and MB. MB was then washed with 100 μL of binding buffer (20 mM Tris-HCl, pH 8.0, 0.5 M NaCl) twice and resuspended in 100 uL of binding buffer. Then, 100 pmol EC1 was added, followed by incubation with mild shaking at room temperature for 1 h (it was found that more than 95% radioactive EC1 was bound to MB). After removing the supernatant, MB was washed twice with 100 μL of binding buffer. This was followed by the addition of 15 μL of UrDNA and 15 μL of 0.5 M NaCl. The mixture was then heated to 45° C. for 2 min and then cooled to room temperature. After incubation at room temperature for 2 h, MB was magnetically separated from the supernatant, washed twice with 100 uL 1× reaction buffer (1×RB; 10 mM HEPES, pH 7.4, 150 mM NaCl, 15 mM MgCl₂, 0.01% Tween 20). The resultant MB-EC1-UrDNA was then resuspended in 100 μL of 1×RB and stored at 4° C.

Preparation of Bacterial Cells.

E. coli K12 MG1655 was used as the intended bacterium and Bacillus subtilis 168 was used as the control. A single colony freshly grown on Luria Broth (LB) agar plate was taken and used to inoculate 2 mL of LB. After shaking at 37° C. for 14 h, the bacterial culture was serially diluted in 10-fold intervals. 100 μL of each diluted solution were plated on the LB agar plates (5 repeats) and cultured at 37° C. for 18 h to obtain the cell counts. For the experiment shown in FIG. 5, the number of E. coli cells used were: 5×10⁷, 5×10⁶, 5×10⁵, 5×10⁴, 5×10³ 500, 50, and 5; the number of B. subtilis cells was 10⁷. For the single cell experiment, six culture tubes containing 2 mL of LB were set up, each of which was inoculated with 100 μL of 0.005 CFU/μL glycerol stock and then incubated at 37° C. A 0.3-mL solution was harvested from each tube at 2, 4, 5, 6, 7, 8, 12, 16 and 24 h. Each cell suspension was centrifuged at 13,000 g for 20 min at 4° C. After the removal of the supernatant, the cells were stored at −20° C. prior to the litmus test.

Litmus Test.

E. coli and B. subtilis cells that were frozen at −20° C. were resuspended in 10 μL of 1× reaction buffer, sonicated for 1 min, put on the ice for 1 min, and sonicated for 1 more min. The cell suspension was then centrifuged at 13,000 g for 5 min at 4° C. The supernatant (10 μL) was mixed with MB-EC1-UrDNA (5 μL of stock described earlier, washed 3 times with 50 μL of 1× reaction buffer) and suspension was incubated at room temperature for 1 h. Then, 90 μL of ddH₂O was added to the reaction vial. Following magnetic separation, 70 μL of the supernatant was transferred into a new reaction tube, followed by the addition of 100 μL of substrate solution (2 M NaCl, 60 mM MgCl₂, 50 mM urea, 1 mM HCl) and 10 μL of 0.04% phenol red. A photograph was taken after a signal-producing time of 0-2 h according to individual experiments for FIGS. 3 and 5.

Measuring pH Changes Using a Hand-Held pH Meter.

A portable FiveGo pH meter equipped with an InLab Ultra-Micro electrode from Mettler Toledo was used to measure pH changes. The cleavage reaction was performed using 10⁷ number of E. coli cells. Following the litmus test procedure as described above, 70 μL of the supernatant was transferred into a new reaction tube followed by addition of 10 μL of 0.04% phenol red. The pH reaction was initiated by addition of 100 μL of the substrate solution. The pH electrode was place directly into the vial and measurements were taken every 30 s for 10 min.

Monitoring pH Changes Using pH Paper Strips.

A pH sensitive paper, Hydrion MicroFine 5.5-8.0, purchased from MicroEssential Laboratories, was used to test the pH of reaction mixtures. The cleavage reaction was performed using 10⁷ number of E. coli cells. Similar to the litmus test outlined above, 70 μL of the supernatant was transferred into a new reaction tube followed by addition of 10 μL of ddH₂O. The pH reaction was initiated by adding 100 μL of the substrate solution. The pH strip was cut into smaller squared pieces that were dipped into the reaction vial at time points of 0, 5, 10, 15, 30, 45, and 60 min to generate FIG. 4B.

### Example 2

### Detection of Uranyl: DNAzyme

Numerous contaminants commonly found in drinking water can go undetected due to their lack of distinguishable appearance or taste. One such contaminant is uranium, a heavy metal that can be most commonly found in the form uranyl in aqueous solutions. Exposure to which can lead to a myriad of negative health effects, including acute kidney failure,^{26,27} developmental disabilities,^{28,29} reproductive disabilities,³⁰ and DNA damage.³¹ The colourimetric assay described herein allows rapid visual determination for the presence of uranium by translating the RNA-cleavage reaction performed by a Uranyl-responsive DNAzyme to the release of a pH-changing enzyme capable of producing a rapid increase in pH. In the presence of the appropriate pH indicator, this pH increase produces a vivid change in colour from yellow to purple.

### Results

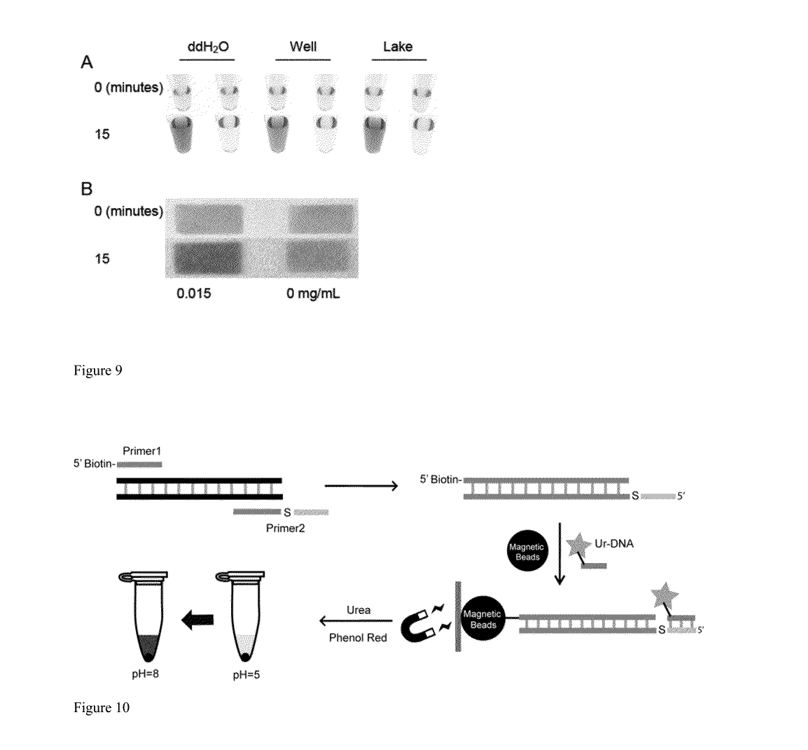

The construct for detection of Uranyl ions was assembled on streptavidin coated magnetic beads using a 5′-biotinylated substrate, a DNA-conjugated urease, and a Uranyl-responsive DNAzyme. In the presence of Uranyl, the RNA was cleaved by the DNAzyme and released the conjugated urease into solution. The released urease was transferred to another vial containing urea, which was hydrolyzed by urease and caused an increase in pH (see FIG. 8). This change in pH was then monitored in the presence of phenol red (See FIG. 9).

### Materials and Methods

Chemical Reagents.

Oligonucleotides were purchased from IDT DNA technologies (Coralville, Iowa, USA). BioMag Streptavidin, Nuclease-free, magnetic beads were purchased from Bangs Laboratories (Fischers, Ind., USA). Uranyl samples were prepared in concentrations corresponding to half of that specified by guidelines provided by the WHO in addition to that of federal regulatory agencies in the United States, and Canada. The WHO and US share a value of 0.03 mg/L, while the Canadian standard is set slightly lower, at 0.02 mg/L.

Well water was obtained through a source from the Ontario Ground Water Association (OGWA), and lake water was obtained from Lake Ontario. The paper based test used Hydrion MicroFine 5.5-8.0 pH paper, which was purchased from MicroEssential Laboratories (Brooklyn, N.Y., USA). All other chemicals and reagents were purchased from Sigma Aldrich (Oakville, Ontario, Canada).

Apparatus and Instruments.

UV/Vis spectra were recorded with a UV/Vis spectrophotometer (Cary) using 1 cm path length quartz cuvettes. Spectral properties assessed at 557 nm at room temperature. Photographs for colour changes were made using a digital camera under manual configuration with 100 ISO and macro activated. The latest version of Photoshop was used to correct white-balance and decrease brightness to −20 for all colourimetric photos.

Assembly of Uranyl Sensor onto Magnetic Beads.

All tubes were pre-washed with 150 μL binding buffer (500 mM NaCl, 20 mM Tris-HCl, pH 7.5, 1 mM MgCl₂, 0.01% v/v Tween 20). All corresponding sequences are listed in Table 2.

100 μL of magnetic beads were washed twice with 150 μL of binding buffer using magnetic separation and resuspended in 90 μL binding buffer prior to addition of 10 μL of BS1 (20 μM). The suspension was allowed to incubate at room temperature for 30 min. The magnetic beads were then washed twice with 150 μL of binding buffer and then resuspended the magnetic beads in 170 μL binding buffer. 30 μL of DE1 (20 μM) was added to the suspension, followed by a brief heating step at 65° C. for 2 min and cooled to room temperature over 10 min. Then 20 pmol of urease linked DNA synthesized in accordance with protocol previously established³² was added and incubated at 37° C. for 10 minutes. The suspension was then allowed to cool to room temperature over 15 min before washing with 100 μL of binding buffer, followed by an additional wash with reaction buffer (300 mM NaCl, 5 mM MES, 0.01% v/v Tween 20). The construct was then resuspended in 100 μL reaction buffer.

TABLE 2
List of sequences used to construct the
Uranyl-responsive sensor

| Name | Labels | Sequence |
|---|---|---|
| BS1 | 5′-Biotin; | TTTTT TTTTT TTACT CACTA |
| SEQ | adenine | TRGGA AGAGA TGGAC GTGTT |
| ID | ribonucleotide | TTTAG GGCAA GTCTC TAATA |
| NO: 10 | (R) | CGCAC GCATC ACA |
| DE1 | None | CACGT CCATC TCTGC AGTCG |
| SEQ | | GGTAG TTAAA CCGAC CTTCA |
| ID | | GACAT AGTGA GT |
| NO: 11 | | |
| LD1 | 5′-NH₂ | TTTTT TTGTG ATGCG TGCGT |
| SEQ | | ATTAG AGACT TGCCC T |
| ID | | |
| NO: 12 | | |

Assay for the Detection of Uranyl.

25 μL of the Uranyl sensor was used for each test sample. For positive tests, well water and lake water samples were spiked with Uranium (0.015 mg/L) and incubated at room temperature for 90 min. When the reaction was complete, 150 μL dH₂O was added and the magnetic bead suspension was placed on a magnetic rack for separation. 20 μL of the supernatant was taken out and transferred to another tube, followed by the addition of 2.5 μL of phenol red and 25 μL substrate solution (2 M NaCl, 60 mM MgCl₂, 50 mM Urea, pH 5.0).

### Example 3

### Detection of Clostridium difficile and Pythium aphanidermatum: PCR

Clostridium difficile (C. difficile) is a Gram-positive, anaerobic, spore-forming bacillus that has been identified as the major cause of antibiotic-associated diarrheal disease and pseudomembranous colitis in humans.³³⁻³⁷ In recent years, both the rate and severity of C. difficile-associated diseases have increased in Canada, the United States and Europe. In Ontario, more than 20 hospitals have declared C. difficile outbreaks since 2011. It is currently estimated that there are 500,000 cases of C. difficile infection (CDI) annually in US hospitals and long-term care facilities, resulting in 14,000 deaths.³⁸ Each case of CDI has been estimated to result in more than $3,600 in health care costs, and overall these costs may exceed $1 billion annually in US.³⁹ The increasing incidence and severity of CDI appear to be linked to the emergence of several new epidemic strains of C. difficile that produces a higher amount of toxins and is more resistant to antibiotics.^{39,40}

An early and accurate detection of C. difficile is important for disease management and infection control. Nucleic acid amplification-based methods, such as polymerase chain reaction (PCR), can be used to target genomic toxin genes to provide rapid and highly sensitive detection. In this study, a novel PCR-urease based test is presented to quickly convert the powerful PCR technique into a simple colourimetric test. Herein, three forward primers were designed that would be able to specifically detect C. difficile and also identify hyper-virulent strains (NAP1/027, NAP7/078).

In addition to targeting C. difficile, the PCR-litmus method was also adopted for the detection of Pythium aphanidermatum. The genus Pythium is regarded as one of the most important groups of soil-borne plant pathogen and close monitoring of this pathogen is required to maintain plant health.⁴¹ Its chronic and ubiquitous infliction on agricultural soil severely affects crop yield and quality by degrading the roots of its host. Constant surveillance of this pathogen is highly desirable. As demonstrated with strain specific identification of C. difficile through the PCR-litmus method, this technique was extended to not only target the presence of Pythium, but also identify the specific species of Pythium in water samples.

### Results

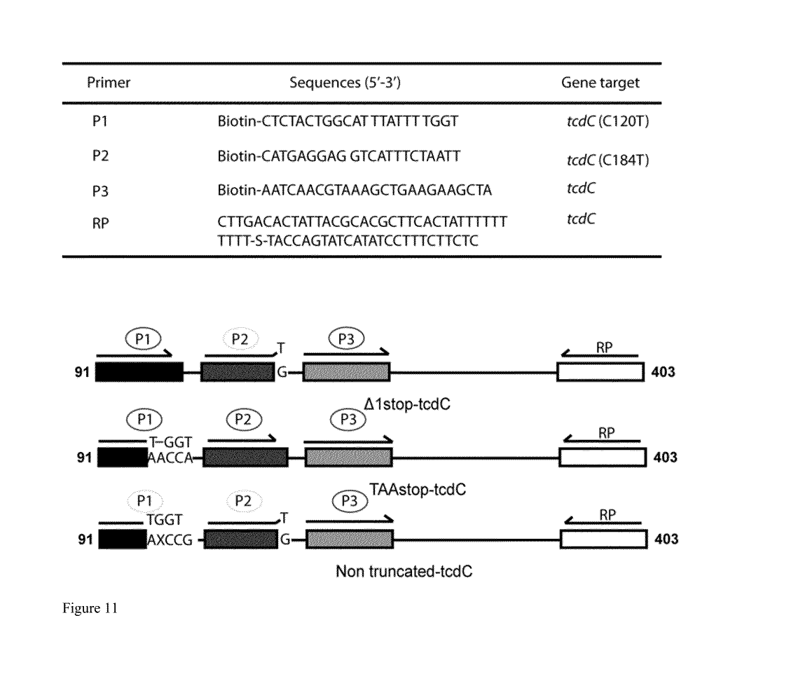

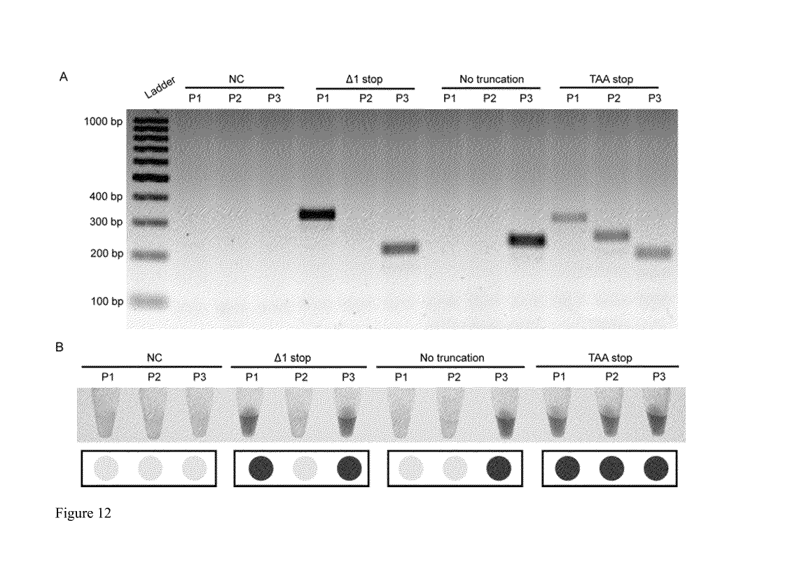

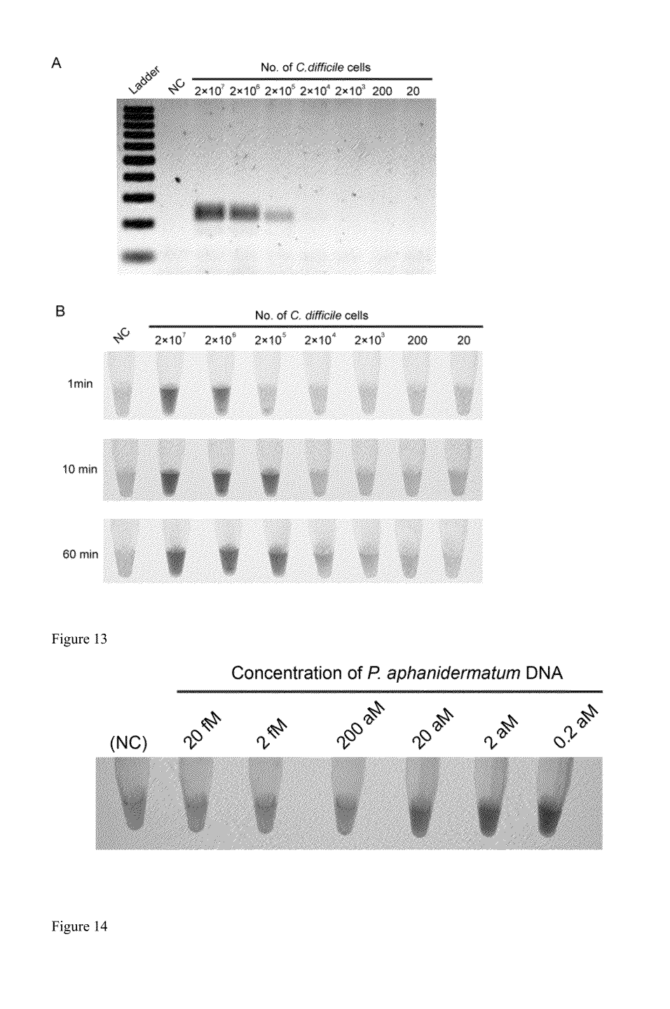

The tcdC gene was targeted with modified forward and reverse primers (See FIG. 11). The forward primer contained a 5′-biotin for subsequent capture of the streptavidin coated magnetic beads. The reverse primer contained an internal triethylene glycol spacer to prevent polymerization of the complementary sequence to the urease conjugated DNA (Ur-DNA). The presence of tcdC generated amplified products that are capable of capturing Ur-DNA and becoming immobilized on the magnetic beads. Addition of urea substrates were hydrolyzed by urease and the pH of the solution increased (FIG. 10). This change in pH was visualized with the addition of phenol red. Detection of the tcdC variants was also shown on gel electrophoresis (FIG. 12). Serial dilutions of C. difficile showed the sensitivity of the assay (FIG. 13).

Primers for P. aphanidermatum were used to demonstrate the ability to colorimetrically detect the presence of the fungus in the same manner as the C. difficile detection (FIG. 14).

Materials and Methods for C. difficile Detection

Chemicals and Reagents.

DNA oligonucleotides were prepared by automated DNA synthesis using standard phosphoramidite chemistry (Integrated DNA Technologies, Coralville, Iowa, USA). All DNA oligonucleotides were purified by 10% denaturing (8 M urea) polyacrylamide gel electrophoresis (dPAGE), and their concentrations were determined spectroscopically. Deoxynucleotide 5′-triphosphates (dNTPs) were purchased from Thermo Scientific (Ottawa, ON, Canada). Thermus thermophilus DNA polymerase was acquired from Biotools. SYBR Gold (10,000× stock in DMSO) was obtained from Life Technologies (Burlington, ON, Canada). Streptavidin coated magnetic beads of 1.5 μm (BioMag-SA) was purchased from Bangs Laboratories Inc. Urease, maleimidobenzoic acid N-hydroxy-succinimide ester (MBS), phenol red were obtained from Sigma-Aldrich. Water was purified with a Milli-Q Synthesis A10 water purification system. All other chemicals were purchased from Bioshop Canada and used without further purification. Urease-DNA was prepared according to a previously reported method.³²

Bacterial Strains and Routine Culture Conditions.

A panel of 14 C. difficile strains were used in this study. The C. difficile strains in this study were obtained from the American Type Culture Collection (Manassas, Va.). C. difficile cultures were grown in cooked meat broth medium, anaerobically, 37° C. in an anaerobic workstation.

Total DNA Extraction.

For crude DNA preparation from C. difficile strains, 200 μL of cultures were spun down (10,000 g, 5 min) in order to remove the culture medium; and the obtained pellets were suspended in 200 μL of 5% Chelex 100 (Bio-Rad) with 0.2 mg protease K. The mixture was then vortexed and incubated at 56° C. for 30 min and 95° C. for 15 min. After centrifugation for 10 min at 10,000 g, the supernatant was transferred into a fresh tube and stored at 4° C. until PCR testing.

Targeting C. difficile tcdC for PCR.

For tcdC gene, a new primer design was conducted with the OligoAnalyzer 3.1 (http://www.idtdna.com/calc/analyzer) after alignment of 26 tcdC fragment gene sequences from PubMLST (http://pubmlst.org/). All primers were checked using the alignments of sequences, and subsequently with the basic local alignment search tool (BLAST; http://www.ncbi.nlm.nih.gov/BLAST/). All primers are listed in FIG. 11.

The PCR mixture (50 μL) contained 4 μL of extracted DNA, 0.5 μM of forward primer and reverse primer, 200 μM each of dNTPs (dATP, dCTP, dGTP and dTTP), 1×PCR buffer (75 mM Tris-HCl, pH 9.0, 2 mM MgCl₂, 50 mM KCl, 20 mM (NH₄)₂SO₄) and 1 unit of Thermus thermophilus (Tth) DNA polymerase. The DNA was amplified using the following thermocycling steps: 94° C. for 5 min; 25 cycles of 94° C. for 1 min, 62° C. for 1 min and 72° C. for 1 min; 72° C. for 5 min.

Urease Litmus Test.

50 μL of the above PCR reaction mixture was incubated with 50 μL of binding buffer (10 mM Tris-HCl, pH 7.5, 3M NaCl, 1 mM MgCl₂, 0.01% Tween 20) along with 10 μL of magnetic beads (MB) for 15 minutes. Then it was placed in a magnetic holder to separate the supernatant from the MB. The MB was then suspended in 100 μL of binding buffer with 1 μL of 1 μM UrDNA. After 15 min of incubation, the MB was washed with 100 μL of binding buffer four times and then resuspended in 70 μL of acetic acid buffer (0.1 mM, pH 5). Then 10 uL of 0.04% phenol red and 100 uL of substrate solution (3 M NaCl, 60 mM MgCl₂, 50 mM urea) were added. Note that this substrate solution should have a starting pH of 5.0. A photograph was taken after a signal-producing time of 0-1 h according to individual experiments.

Sensitivity Test.

A single colony of different strains of C. difficile (ATCC1803, ATCC1871, ATCC1875) from an anaerobic cooked meat broth agar plate was taken and cultured in 5 mL of cooked meat broth medium overnight. The bacterial culture was then diluted in 10-fold intervals seven times with cooked meat broth medium; 100 μL of 10⁻⁵, 10⁻⁶ and 10⁻⁷ dilutions were placed on a cooked meat broth plate and cultured for colony development in order to calculate the number of colony-forming units (CFU) for each dilution. Diluted cultures were then used for DNA extraction and PCR reaction.

Materials and Method for Pythium aphanidermatum

DNA Template Dilutions.

Five 10-fold serial dilutions were performed using an initial 1 μM solution of Pythium aphanidermatum ITS1 DNA template (5′-GTA GTC TGC CGA TGT ATT TTT CAA ACC CAT TTA CCT AAT ACT GAT CTA TAC TCC AAA AAC GAA AGT TTA TGG TTT TAA TCT ATA ACA ACT TTC AGC AGT GGA-3′ (SEQ ID NO:13)) to create solutions with concentrations of 100 fM, 10 fM, 1 fM, 100 aM, and 10 aM. All dilutions were made with ddH₂O.

PCR Amplification.

PCR products of the ITS1 region of Pythium aphanidermatum (102 bp) were obtained using 5′-biotin labelled FP (5′-biotin-GTA GTC TGC CGA TGT ATT-3′ (SEQ ID NO:14)) and 5′-urease binding site labelled RP (5′-CGT GAC CTA CCT TAC CTC TTG ACC TTG AAA AAA /iSp9/ TCC ACT GCT GAA AGT TG (SEQ ID NO:15); /iSp9: internal triethylene glycol spacer). The PCR reaction (50 μL) contained 0.5 μM of each primer, 2.5 units of Taq DNA Polymerase (GenScript), 0.2 mM dNTP mixture (G-Biosciences), 1×Taq Buffer (GenScript) (50 mM KCl, 10 mM Tris-HCl pH 9.0, 1.5 mM MgCl₂, and 0.1% Triton X-100), and 1 μL of the DNA template concentrations described above. The reactions was conducted using a DNA Robocycler Gradient 96 (Stratagene) and amplification conditions were an initial denaturation at 94° C. for 5 min, followed by 35 cycles of denaturation at 94° C. for 45 sec, annealing at 54° C. for 45 sec, and extension at 72° C. for 45 sec with a final extension at 72° C. for 5 min. The PCR product size was examined by electrophoresis in a 2% agarose RA (Amresco) gel. Gels were stained with SYBR Safe DNA Gel Stain (Invitrogen) and scanned using a Typhoon 9410 Multipurpose Scanner from GE Healthcare.

Urease Litmus Test.

Urease-DNA was prepared according to a previously reported method.³² 10 μL streptavidin-coated magnetic beads (MB) (BioMag-SA) was washed with 100 μL of binding buffer (20 mM Tris-HCl, pH 8.0, 0.5 M NaCl) twice, using a magnet holder to separate the MB from the supernatant and removing the supernatant after each wash. 50 μL of PCR product and 50 μL of binding buffer were then added to the MB and the PCR product was allowed to incubate with the MB for 15 min. After the incubation, the MB was magnetically separated from the supernatant and washed once with 100 μL of binding buffer. This was followed by the addition of 1 μL of 5′-amino conjugated UrDNA (0.5 μM; DNA sequence: 5′-H₂N-AAA AAA CAA GGT CAA GAG GTA AGG TAG GTC ACG-3′ (SEQ ID NO:16)) along with 100 μL of binding buffer and the mixture was allowed to react at room temperature for 15 min. After the second reaction, the MB was again magnetically separated from the supernatant and washed four times with 100 μL of Binding Buffer. Following the washes, 70 μL of acetic acid buffer (1 mM, pH 5) and 10 μL of 0.04% phenol red were added to the MB, followed by the addition of 100 μL of substrate solution (2 M NaCl, 60 mM MgCl₂, 50 mM urea, 1 mM HCl). The solution was mixed vigorously by shaking. Photographs were taken at 15 min intervals starting at 0 min up to 1 hour.

### Example 4

### Detection of ATP Using Ligation

ATP is a ubiquitously occurring molecule in nature, playing a multifaceted role in cellular life in a broad array of organisms, from bacteria to mammals. In addition to its role as a component of the genetic code, ATP is commonly used as a cofactor in many enzyme-catalyzed reactions, most notably as a source of energy.^{42,43} It may serve a role as part of enzymatic reaction mechanisms, or as a source of phosphate for kinase-catalyzed reactions.^{42,44,45} As a result of its regular utilization and ubiquity, it can be used as an effective biomarker of bacterial growth and contamination, as its concentration has been previously shown to directly correlate with bacterial growth.^{46,47} Detection of ATP can be carried out through molecular recognition by an ATP-dependent enzyme or functional nucleic acid.^{45,48} However, current approaches do not offer the simplicity or cost effectiveness necessary for large-scale use in developing regions. The luciferase-luciferin system, which is the most commonly used method for ATP detection, makes use of the highly unstable luciferase enzyme, and requires a luminometer for signal detection.^{45,49} Other systems utilizing ligases or functional nucleic acids generate fluorescent or electrochemical signals that are not amenable to use in low skill environments or impoverished regions.⁵⁰ These obstacles can effectively be overcome by the use of a colourimetric assay, but current colourimetric ATP detection systems, such as those based on polythiophene derivatives and gold nanoparticles have failed to demonstrate the sensitivity needed for reliable bacterial detection.⁵¹⁻⁵³

### Results

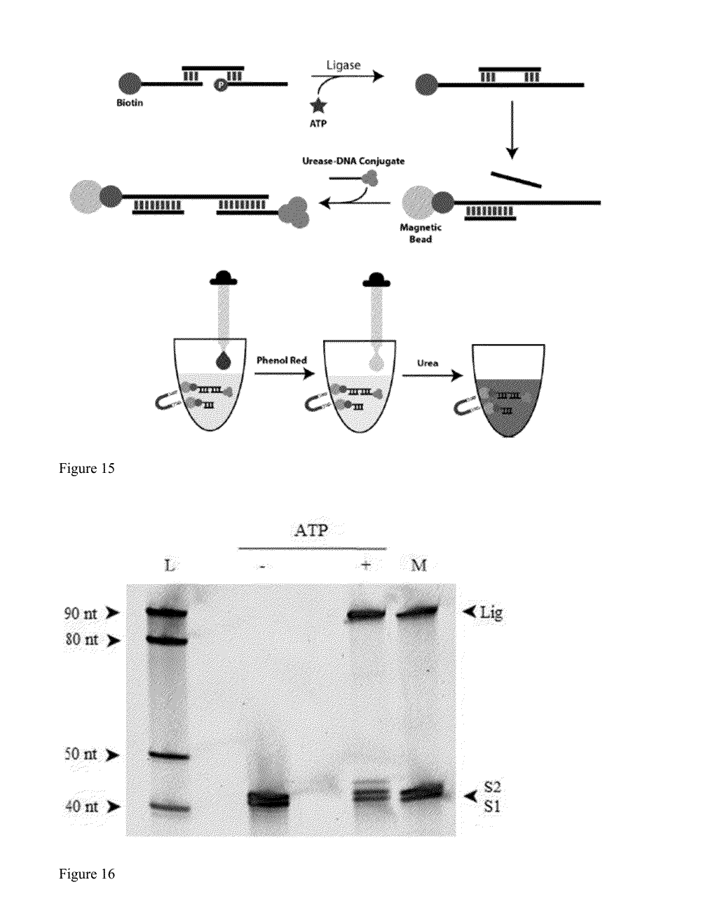

In this study, the present inventors developed a simple, portable, and reliable biosensor for ATP. This sensor relies on an ATP-dependent ligation catalyzed by T4 DNA Ligase.⁴⁴ The enzyme-catalyzed reaction links a biotinylated DNA strand to a DNA strand capable of duplexing with a urease-conjugated DNA. Using streptavidin-coated magnetic beads, the urease coupled DNA duplex was isolated from the bulk solution. Addition of urea resulted in urease catalyzed hydrolysis of the substrate, generating a pH change that was colourimetrically observed through the use of a pH indicator dye (FIG. 15).

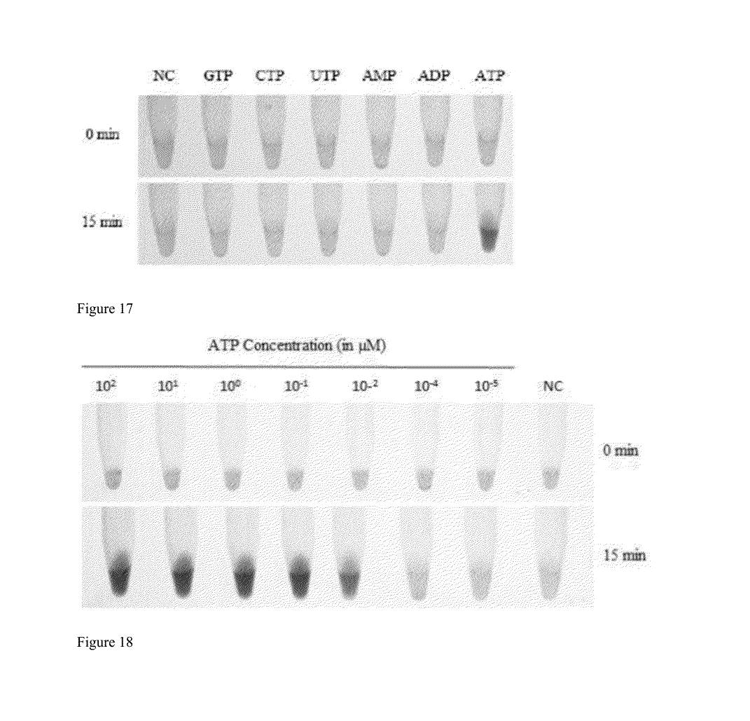

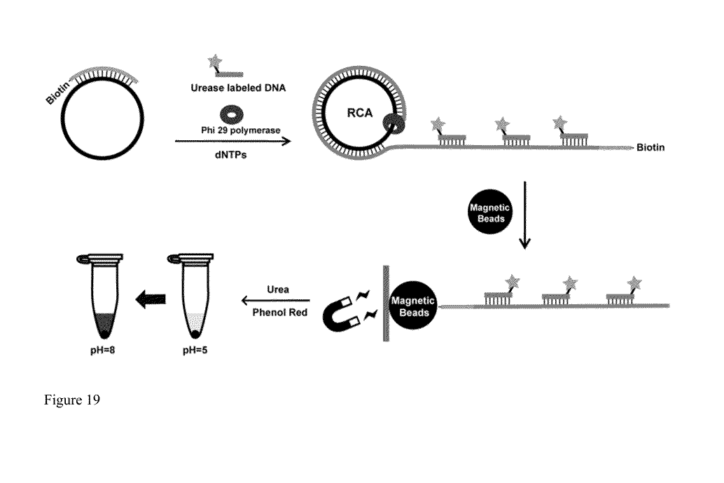

In particular, T4 DNA ligase catalyzed the ATP-dependent ligation of the 5′-phosphorylated sequence to a biotinylated DNA oligonucleotide. A DNA strand complimentary to the biotinylated strand was used to remove the ligation template and any residual unligated phosphorylated strands, as well as to prevent weak association between UrDNA and the biotinylated strand. The phosphorylated sequence contained a 26 nt region complementary to the urease-conjugated DNA sequence. When these sequences were successfully ligated in the presence of ATP, streptavidin-coated magnetic beads were used to isolate UrDNA captured by the ligation product through interaction with the 5′ biotin label. Finally, addition of a pH indicator dye and urea generated a colourimetric signal in the form of a shift from bright yellow to red in the presence of UrDNA (FIG. 15). FIG. 16 shows the results of ligation of S1 and S2 (SEQ ID NOs: 19 and 20 from Table 3) in the presence of ATP as shown using electrophoresis. FIGS. 17 and 18 show the results of the colourimetric test showing the specificity and sensitivity of the assay.

### Materials and Methods

DNA Oligonucleotides & Chemical Reagents.

The magnetic beads used in all experiments were the BioMag® Streptavidin Nuclease-free 1.5 μm diameter magnetic beads (Bangs Laboratories Inc.). The magnetic rack used was the 6-tube Magnetic Separation Rack (New England Biolabs). Images were captured using a Canon PowerShot G11 camera. Fluorescent gels were scanned and imaged by a Typhoon 9410 Multipurpose Scanner (GE Healthcare). DNA oligonucleotides were purchased from Integrated DNA Technologies. ADP and AMP were purchased from Sigma-Aldrich. Reagents used for the conjugation of Urease to DNA were purchased from the sources used previously.³² SYBR® Gold Nucleic Acid Gel Stain was purchased from Life Technologies. All other reagents were purchased from ThermoFischer Scientific unless otherwise specified. The DNA oligonucleotides used for these experiments in this study are shown in the Table 3 below.

TABLE 3
List of sequence design for ATP detection

| Name | Label | Sequence (5′ → 3′) |
|---|---|---|
| LD1 | 5′ NH2 | TTTTT TAGTG AAGCG TGCGT AATAG |
| SEQ ID | | TGTCA AG |
| NO: 17 | | |
| FL1 | 5′ Biotin | TTTTT TTTTT TTCTA TGAAC TGACT |
| SEQ ID | | ATGAC CTCAC TACCA AGAAC GCTTA |
| NO: 18 | | CAATG ACACT CCCTT GACAC TATTA |
| | | CGCAC GCTTC ACT |
| S1 | 5′ Biotin | TTTTT TTTTT TTCTA TGAAC TGACT |
| SEQ ID | | ATGAC CTCAC TACCA AG |
| NO: 19 | | |
| S2 | 5′ | AACGC TTACA ATGAC ACTCC CTTGA |
| SEQ ID | Phosphate | CACTA TTACG CACGC TTCAC T |
| NO: 20 | | |
| Template | No Label | ATTGT AAGCG TTCTT GGTAG TGAG |
| SEQ ID | | |
| NO: 21 | | |
| B1 | No Label | CTTGG TAGTG AGGTC ATTGT CAGTT |
| SEQ ID | | CATAG |
| NO: 22 | | |

Positive Colour Test with Full Length Construct.

Urease was conjugated to a DNA sequence as previously described³² generating a urease-DNA conjugated macromolecule (UrDNA). To determine the quantity of UrDNA to be used in subsequent assays, serial 1:10 dilutions were prepared. 1 μL of FL1 (20 μM) was incubated with 15 μL of nuclease-free streptavidin coated magnetic beads in 75 μL binding buffer (10 mM Tris pH 7.5, 3 M NaCl, 1 mM MgCl₂, 0.01% Tween 20) for 15 minutes at room temperature. 1 μL of UrDNA at each dilution was applied to the solution and allowed to incubate at room temperature for 15 minutes. The magnetic beads were then separated from solution on a magnetic rack and washed four times in binding buffer. The magnetic beads were resuspended in 70 μL 1 mM acetic acid buffer and 10 μL of 0.04% phenol red. 100 μL substrate solution (2M NaCl, 60 mM MgCl₂, 50 mM Urea) was added to the suspension, and the resulting mixtures were observed for colour change.

Detection of ATP.

The ligation reactions was prepared by mixing the following: 1 μL of S1 (15 μM), 1 μL of S2 (30 μM), 1 μL of template (20 μM), 1 μL of 10× ligation buffer (400 mM Tris-HCl pH 7.8, 100 mM MgCl₂, 100 mM dithiothreitol), and 1 μL 5 U/μL T4 DNA ligase. For the specificity test, ATP was added to a final concentration of 100 nM. For the sensitivity test, the final ATP concentration for each positive sample was 100 uM, 10 uM 1 uM, 100 nM, 10 nM, 1 nM, and 0.1 nM. The total volume for all ligation reactions were topped to a total volume of 10 uL with ddH₂O and incubated for 30 minutes at 25° C.

In a separate tube, 10 μL of magnetic beads were washed with 100 μL binding buffer and resuspended in 75 μL binding buffer. Completed ligation reactions and 1 μL of 60 μM B1 was added to the suspended magnetic beads and incubated for 15 minutes at 25° C. These solutions were then washed and resuspended in 75 μL binding buffer, and a further 1 μL of 60 μM B1 was added. Following a second incubation for 15 minutes at 25° C., the magnetic bead suspension was washed and resuspended in 75 μL binding buffer. UrDNA was added, and the solution was incubated for 15 minutes at 25° C. This solution was then washed twice with 100 μL of binding buffer and twice with 100 μL of 1 mM acetic acid buffer at pH 5.0. The washed magnetic beads were resuspended in 70 μL acetic acid buffer and 10 μL of 0.04% phenol red. 100 μL of substrate was added to the suspension, and the resulting mixtures were observed for colour change and absorbance.

### Example 5

### Project Rolling Circle Amplification

Rolling circle amplification (RCA) is a widely used method for DNA amplification. It is an isothermal reaction that generates extremely long DNA molecules from a single-stranded circular DNA sequence.^{54,55} This unique reaction relies on special DNA polymerases such as phi29, which makes repetitive rounds of DNA replication over the circular template. The reaction is initiated in the presence of a short DNA primer and utilizes deoxyribonucleotides 5′triphosphates (dNTPs) as the building blocks. RCA is often exploited as a signal amplification technique as thousands of synthesized tandem DNA repeats can be generated from a single primer-template recognition event.⁵⁶⁻⁵⁸ Additionally, the reactions can be carried out at room temperature, which allows this method to not rely on large expensive equipment. With these unique advantages, RCA can be a powerful method in creating highly sensitive biosensors.

### Results

To expand on the previously developed urease colourimetric test as described in Example 1 and in Tram et al.³² incorporation of the RCA technique can be used as a means to increase the sensitivity of DNA detection. In this test, the fold-enhancement in signal between RCA and standard DNA capture was compared. Through the process of RCA, the increased number of repeated units that allow for complementary binding of a DNA-conjugated urease increased the detection sensitivity by 100-fold over the unamplified method. More capture of DNA-conjugated urease allowed greater rates of urea hydrolysis, which in turn changed the pH of the solution faster. This change in pH was easily visualized using phenol red as a pH indicator.

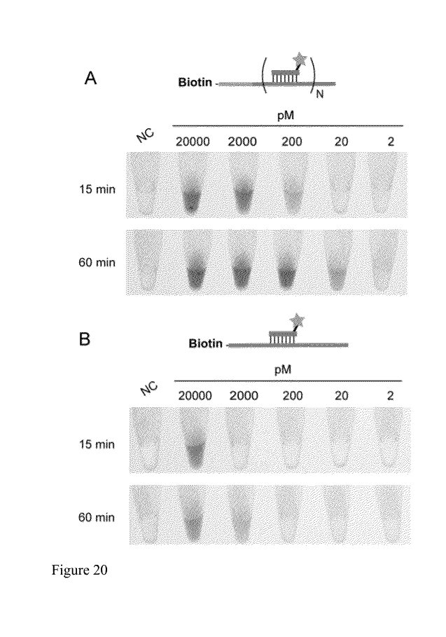

In particular, the reaction is initiated with a 5′-biotinylated primer that hybridizes to the circular template. Addition of phi29 polymerase and dNTPs generated a long single stranded DNA with many repeating DNA units. These repeats hybridized with the Ur-DNA present in solution and addition of magnetic beads captured the entire RCA product along with the Ur-DNA. This complex catalyzed the hydrolysis of urea and raised the pH of the solution, which was monitored by a pH indicator (FIG. 19). FIG. 20 shows that the colourimetric test allowed for more than 100-fold signal enhancement.

### Materials and Method

Enzymes, Chemicals and Other Materials.

T4 DNA ligase, T4 polynucleotide kinase (PNK), ATP and deoxynucleoside 5′-triphosphates (dNTPs) were purchased from Thermo Scientific (Ottawa, ON, Canada). Phi29 DNA polymerase was purchased from Lucigen (Mississauga, ON, Canada). SYBR Gold (10,000× stock in DMSO) was obtained from Life Technologies (Burlington, ON, Canada). Water was purified with a Milli-Q Synthesis A10 water purification system and then autoclaved. 10×PBS (pH 7.4) was purchased from BioShop Canada (Burlington, ON. Canada). Streptavidin coated magnetic beads of 1.5 um (BioMag-SA) was purchased from Bangs Laboratories Inc (Burlington, ON, Canada). All other materials were purchased from Sigma-Aldrich (Oakville, ON, Canada).

Synthesis and Purification of Oligonucleotides.

All oligonucleotides were purchased from Integrated DNA Technologies (IDT) and their sequences are listed in the Table 4 below. The oligonucleotide DNA sequences were all purified using 10% denaturing (8 M urea) polyacrylamide gel electrophoresis (dPAGE). The DNA was then eluted from the gel overnight using 500 uL of elution buffer (0.2 M NaCl, 0.01 M Tris pH 7.5, 1 mM EDTA pH 8.0). After the elution, all the sequences underwent ethanol precipitation for further purification before measuring their concentrations spectroscopically using the UVspec meter.

TABLE 4
List of sequence design for RCA-litmus test

| Name | Label | Sequence (5′ → 3′) |
|---|---|---|
| CT | No Label | AAGGA GTGAA GCGTG CGTAA TAGTG |
| SEQ ID | | TCAAG GAATT CAATC A |
| NO: 23 | | ACGTAAAGCTGAAGAAGCT |
| LT | No Label | CTCCT TAGCT TCTTC A |
| SEQ ID | | |
| NO: 24 | | |
| UrD | 5′-NH₂, | TTTTT TAGTG AAGCG TGCGT AATAG |
| SEQ ID | 3′- | TGTCA AG |
| NO: 25 | Inverted T | |
| BP | 5′-Biotin | CTCCT TAGCT TCTTC A |
| SEQ ID | | |
| NO: 26 | | |
| BM | 5′-Biotin | TCTTC AGCTT TACGT TGATT GAATT |
| SEQ ID | | CCTTG ACACT ATTAC GCACG CTTCA |
| NO: 27 | | CTCCT TAGCT |

Synthesis of Circular DNA Templates.

Circular DNA templates were prepared from 5′-phosphorylated linear DNA oligonucleotides through template-assisted ligation with T4 DNA ligase. Linear DNA oligonucleotide (CT) was phosphorylated as follows: a reaction mixture (50 μL) was made to contain 1 nM CT, 20U PNK (U: unit), 1×PNK buffer A (50 mM Tris-HCl, pH 7.6 at 25° C., 10 mM MgCl₂, 5 mM DTT, 0.1 mM spermidine), and 2 mM ATP. The mixture was incubated at 37° C. for 30 min, followed by heating at 90° C. for 5 min. The circularization reaction was conducted in a volume of 400 μL, produced by adding 306 μL of H₂O and 2 μL of a DNA template (LT, 500 μM) to the phosphorylation reaction mixture above. After heating at 90° C. for 3 min and cooling down at room temperature (RT) for 10 min, 40 μL of 10×T4 DNA ligase buffer (400 mM Tris-HCl, 100 mM MgCl₂, 100 mM DTT, 5 mM ATP, pH7.8 at 25° C.) and 2 μL of T4 DNA ligase (5 U/μL) were added. This mixture was incubated at RT for 2 h before heating at 90° C. for 5 min to deactivate the ligase. The ligated circular DNA molecules were concentrated by standard ethanol precipitation and purified by 10% dPAGE. The concentration of the circular DNA template was determined spectroscopically.

RCA Reaction.

The RCA reaction was performed in a total volume of 50 μL. 1 μL of 0.1 μM circular DNA template (CT) (the final concentration=2 nM) was mixed with BP (to obtain final concentrations of 20 nM, 2 nM, 200 pM, 20 pM, or 2 pM), 5 μL of 2 mM each of dGTP, dATP, dTTP and dCTP (the final concentration=0.2 mM each), 5 μL 10×RCA buffer (50 mM Tris-HCl, 10 mM (NH₄)₂SO₄, 4 mM dithiothreitol, 10 mM MgCl₂, pH 7) and 37.5 μL of H₂O. After heating at 90° C. for 3 min, the solution was cooled to room temperature for 10 min. 0.5 μL of 10 U/μL phi29 DNA polymerase and 1 μL of 1 μM were then added, followed by incubation at 30° C. for 120 min.

Colourimetric Test of RCA and Unamplified DNA Capture.

The protocol uses the Ur-DNA conjugation published previously.³² Once the product was amplified from RCA (total volume of 50 uL), it was added to a tube containing 10 uL of magnetic beads along with 50 uL of binding buffer (1×BB; 1 mM HEPES, pH 7.4, 150 mM NaCl, 15 mM MgCl₂, 0.01% Tween 20) and the suspension was incubated for 15 minutes at room temperature. For the unamplified experiment, a suspension of 10 uL of magnetic beads, 5′-biotin sequence BM (20 nM, 2 nM, 200 pM, 20 pM, or 2 pM) in 50 uL of binding buffer was incubated for 15 minutes at room temperature. After magnetic separation, the solutions were removed and the beads were washed with 100 uL of binding buffer. That solution was removed once again and 100 uL of fresh binding buffer was added along with 1 uL of the conjugated Ur-DNA. This solution was incubated at room temperature for 15 minutes and was then washed 4 times with binding buffer. Then 70 uL of acetic acid and 10 uL of 0.04% phenol red and 100 uL of substrate solution (3 M NaCl, 60 mM MgCl₂, 50 mM urea, 1 mM HCl) was added to the magnetic beads.

### Example 6

### Capture of Viral Oligonucleotide

A simple colourimetric test for detecting oligonucleotides is presented herein.

### Results

Target oligonucleotides were used as a bridging sequence to link a 5′-biotinylated sequence to a urease conjugated-DNA. This quick one-pot hybridization reaction allowed for the capture of urease, a pH-changing enzyme. The presence of urease hydrolyzed its cognate substrate urea to ammonia, which resulted in an increase of pH. This pH change could then be readily observed using a known pH indicator such as phenol red.

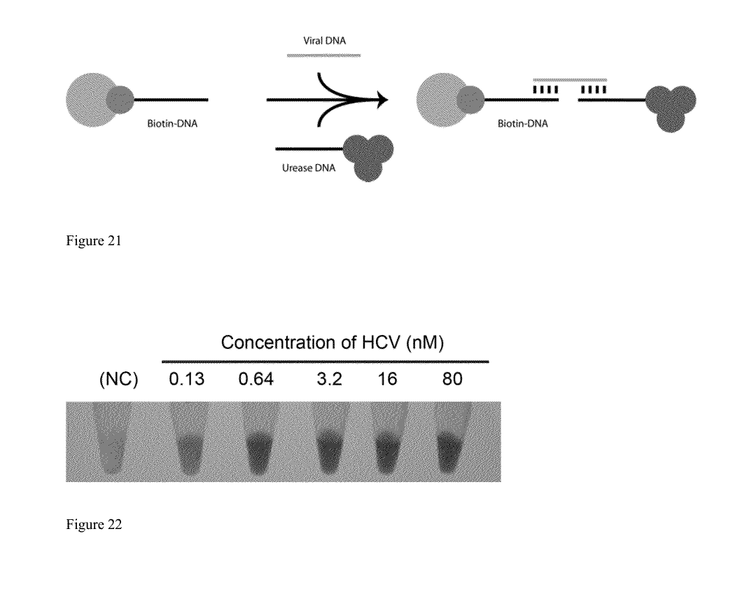

In particular, a 5′-biotinylated DNA recognized the target oligonucleotide through Watson-Crick base pairing interaction. Addition of a matching Urease-DNA component completed the functional complex that was then captured by the magnetic beads. The presence of urease in this complex hydrolyzed the urea substrate and raised the pH of the solution. This pH change was visualized using a pH indicator such as phenol red (FIG. 21). FIG. 22 shows the results of detection of Hepatitis C viral (HCV) DNA.

### Materials and Methods

The magnetic beads used in the experiments were the BioMag® Streptavidin Nuclease-free 1.5 μm diameter magnetic beads (Bangs Laboratories Inc.). The magnetic rack used was the 6-tube Magnetic Separation Rack (New England Biolabs). Images were captured using a Canon PowerShot G11 camera. DNA oligonucleotides were purchased from Integrated DNA Technologies. All other reagents were purchased from ThermoFischer Scientific unless otherwise specified.

HCV Template Dilution.

Four 5-fold serial dilutions were performed using an initial 4 pM solution of HCV DNA template (5′-AGT CCA CCG TGT CGT CTG CT-3′ (SEQ ID NO:28)) to create solutions of concentrations 4 pM, 800 nM, 160 nM, 32 nM, 6.4 nM. All dilutions were prepared with dH₂O.

Litmus Test for HCV DNA.

Urease was conjugated to a DNA sequence as previously described.³² 2 μL 3′-labelled Biotin substrate (2.5 μM, 5′-ACG GTG GAC TTT TTT TTT TTT T-Biotin-3′ (SEQ ID NO:29)), 1 μL of 5′-amino conjugated UrDNA (4 pM, 5′-H₂N-TTT TTT TTT AGC AGA CGA C-3′(SEQ ID NO:30)) and 1 μL of the HCV sequence at the given concentrations described above, were mixed and adjusted to a final volume of 50 μL with 1×PBS Buffer. The solution was incubated at 60° C. for 2 min followed by room temperature for 30 min. Separately, 10 μL of streptavidin-coated magnetic beads (MB) (BioMag-SA) was washed with 100 μL of Binding Buffer (20 mM Tris-HCl, pH 8.0, 0.5 M NaCl) twice, using a magnet holder to separate the MB from the supernatant and removing the supernatant after each wash. The 50 μL mixture described above was then added to the MB and allowed to react at room temperature for 20 min. After the reaction, the MB was magnetically separated from the supernatant, washed twice with 100 μL of Binding Buffer then twice with 1 mM acetic acid buffer, pH 5.0. Following the washes, 70 μL of acetic acid buffer and 10 μL of 0.04% phenol red were added to the MB, followed by the addition of 100 μL of substrate solution (2 M NaCl, 60 mM MgCl₂, 50 mM Urea, 1 mM HCl). The solution was mixed vigorously by shaking. Photographs were taken to record the colour change.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

[1] A. S. Daar, H. Thorsteinsdottir, D. K. Martin, A. C. Smith, S. Nast, P. A. Singer, Nat. Genet. 2002, 32, 229-232.
[2] J. D. Newman, A. P. Turner, Biosens. Bioelectron. 2005, 20, 2435-2453.
[3] A. P. Turner, Chem. Soc. Rev. 2013, 42, 3184-3196.
[4] J. B. Sumner, D. B. Hand, J. Am. Chem. Soc. 1929, 51, 1255-1260.
[5] P. A. Karplus, M. Pearson, R. P. Hausinger, Acc. Chem. Res. 1997, 30, 330-337.
[6] B. E. Dunn, G. P. Campbell, G. I. Perez-Perez, M. J. Blaser, J. Biol. Chem. 1990, 265, 9464-9469.
[7] G. W. Stemke, J. A. Robertson, M. P. Nhan, Can. J. Microbiol. 1987, 33, 857-862.
[8] D. L. Clemens, B. Y. Lee, M. A. Horwitz, J. Bacteriol. 1995, 177, 5644-5652.
[9] (a) C. Tuerk, L. Gold, Science 1990, 249, 505-510; (b) A. D. Ellington, J. W. Szostak, Nature 1990, 346, 818-822.
[10] (a) L. C. Bock, L. C. Griffin, J. A. Latham, E. H. Vermaas, J. J. Toole, Nature 1992, 355, 564-566; (b) A. D. Ellington, J. W. Szostak, Nature 1992, 355, 850-852.
[11] G. F. Joyce, Annu. Rev. Biochem. 2004, 73, 791-836.
[12] M. Famulok, J. S. Hartig, G. Mayer, Chem. Rev. 2007, 107, 3715-3743.
[13] (a) T. Hermann, D. J. Patel, Science 2000, 287, 820-825; (b) E. N. Brody, L. J. Gold, J. Biotechnol. 2000, 74, 5-13; (c) M. Famulok, G. Mayer, M. Blind, Acc. Chem. Res. 2000, 33, 591-599; (d) G. Mayer, Angew. Chem. Int. Ed. 2009, 48, 2672-2689.
[14] (a) Y. Li, R. R. Breaker, Curr. Opin. Struct. Biol. 1999, 9, 315-323; (b) D. A. Baum, S. K. Silverman, Cell. Mol. Life Sci. 2008, 65, 2156-2174; (c) S. K. Silverman, Angew. Chem. Int. Ed. 2010, 49, 7180-7201.
[15] (a) J. Liu, Y. Lu, Angew. Chem. Int. Ed. 2007, 46, 7587-7590; (b) J. H. Lee, Z. D. Wang, J. W. Liu, Y. Lu, J. Am. Chem. Soc. 2008, 130, 14217-14226; (c) P. W. Wu, K. V. Hwang, T. Lan, Y. Lu, J. Am. Chem. Soc. 2013, 135, 5254-5257; (d) Y. Xiang, Y. Lu, Nat. Chem. 2011, 3, 697-703; (e) P. J. J. Huang, J. Liu, Anal. Chem. 2014, 86, 5999-6005.
[16] (a) Y. C. Huang, B. X. Ge, D. Sen, H. Z. Yu, J. Am. Chem. Soc. 2008, 130, 8023-8029; (b) Y. T. Tang, B. X. Ge, D. Sen, H. Z. Yu, Chem. Soc. Rev. 2014, 43, 518-529.
[17] (a) N. K. Navani, Y. Li, Curr. Opin. Chem. Biol. 2006, 10, 272-281. (b) J. Liu, Z. Cao, Y. Lu, Chem. Rev. 2009, 109, 1948-1998; (c) H. Q. Zhang, F. Li, B. Dever, X. F. Li, X. C. Le, Chem. Rev. 2013, 113, 2812-2841.
[18] (a) R. R. Breaker, G. F. Joyce, Chem. Biol. 1994, 1, 223-229; (b) S. W. Santoro, G. F. Joyce, Biochemistry 1998, 37, 13330-13342; (c) D. Y. Wang, B. H. Lai, D. Sen, J. Mol. Biol. 2002, 318, 33-43; (d) S. H. Mei, Z. Liu, J. D. Brennan, Y. Li, J. Am. Chem. Soc. 2003, 125, 412-420; (e) M. M. Ali, Y. Li, Angew. Chem. Int. Ed. 2009, 48, 3512-3515; (f) S. K. Silverman, Nucleic Acids Res. 2005, 33, 6151-6163; (g) K. Schlosser, Y. Li, Chem. Biol. 2009 16, 311-322.
[19] B. I. Haukanes, C. Kvam, Nat. Biotech. 1993, 11, 60-63.
[20] J. E. Brinchmann, F. Vartdal, G. Gaudernack, G. Markussen, S. Funderud, J. Ugelstad, E. Thorsby, Clin. Exp. Immunol. 1988, 71, 182-186.
[21] L. Johansen, K. Nustad, T. B. Orstavik, J. Ugelstad, A. Berge, T. J. Ellingsen, J. Immunol. Methods. 1983, 59, 255-264.
[22] O. S. Gabrielsen, E. Homes, L. Korsnes, A. Ruet, T. B. Oyen, Nucleic Acids Res. 1989, 17, 6253-6267.
[23] C. Albretsen, K. H. Kalland, B. I. Haukanes, L. S. Havarstein, K. Kleppe, Anal. Biochem. 1990, 189, 40-50.
[24] M. M. Ali, S. D. Aguirre, H. Lazim, Y. Li, Angew. Chem. Int. Ed. 2011, 50, 3751-3754.
[25] S. D. Aguirre, M. M. Ali, B. J. Salena, & Y. Li, Biomolecules 2013, 3, 563-577.
[26] S. Lu, F. Y. Zhoa, Health Phys. 1990, 58, 619-623.
[27] M. L. Zamora, B. L. Tracy, J. M. Zielinski, D. P. Meyerhof, M. A. Moss, Toxicol. Sci. 1998, 43, 68-77.
[28] J. L. Domingo, A. Ortego, J. L. Paternain, J. Corbella, Ecotoxicol. Environ. Saf 1989, 44, 395-398.
[29] L. M. Shields, W. H. Wiese, B. J. Skipper, B. Charley, L. Benally, Health Phys. 1992, 63, 542-551.
[30] J. L. Paternain, H. L. Domingo, A. Ortega, J. M. Lloblet, Ecotoxicol. Environ. Saf 1989, 17, 291-296.
[31] R. Zaire, C. S. Griffin, P. J. Simpson, D. G. Papworth J. R. Savage, S. Armstrong, S., et al., Mutat. Res. 1996, 371, 109-113.
[32] K. Tram, P. Kanda, B. J. Salena, S. Huan, Y. Li, Angew. Chem. 2014, 126, 13013-13016.
[33] J. G. Bartlett, N. Moon, T. W. Chang, N. Taylor, A. B. Onderdonk. Gastroenterology, 1978. 75, 778-782.
[34] H. E. Larson, A. B. Price, P. Honour, S. P. Borriello, Lancet 1978, 1, 1063-1066.
[35] J. G Bartlett, Clin. Infect. Dis. 1994, 18, S265-S272.
[36] M. Rupnik, M. H. Wilcox. D. N. Gerding, Nature Rev. 2009, 7, 526-536.
[37] M. Sebaihia, B. W. Wren, P. Mullany, N. F. Fairweather, N. Miton, et al. Nature Genet. 2006, 38, 779-786.
[38] Antibiotic resistance threats in the United States, 2013. http://www.cdc.gov/drugresistance/threat-report-2013/
[39] L. C. McDonald, G. E. Killgore, A. Thompson, R. C. Owens Jr, S. V. Kazakova, S. P Sambol, S. Johnson, D. N. Gerding, N. Engl. J. Med. 2005, 353, 2433-2441.
[40] B. Hubert, V. G. Loo, A. M. Bourgault, L. Poirier, A. Dascal, E. Fortin, M. Dionne, M. Lorange, Clin. infect. dis. 2007, 44, 238-244.
[41] K. Schroeder, Plant dis. 2013, 97, 4-20.
[42] R. Alberty, J. Chem. Educ. 1969, 46, 713.
[43] J. Strominger, L. Heppel, E. Maxwell, Biochimica et Biophysica Acta, 1959, 32, 412-421.
[44] B. Weiss, C. Richardson, Proc. Natl. Acad. Sci. USA 1967, 57, 1021-1028.
[45] G. Lyman, J. DeVincenzo, Anal. Biochem. 1967, 21, 435-443.
[46] S. Jin, S. Guo, P. Zuo, B. Ye, Biosens. Bioelectron. 2015, 63, 379-383.
[47] S. Imai, M. Nakazawa, M. Role Of Adenosine And Adenine Nucleotides In The Biological System; Elsevier: Amsterdam, 1991.
[48] C. Ispas, G. Crivat, S. Andreescu, Anal. Lett. 2012, 45, 168-186.
[49] W. McElroy, A. Chase, J. Cell. Physiol. 1951, 38, 401-408.
[50] P. Yager, T. Edwards, E. Fu, K. Helton, K. Nelson, M. Tam, B. Weigl, Nature 2006, 442, 412-418.
[51] C. Li, Numata, M. Takeuchi, S. Shinkai, Angew. Chem. 2005, 117, 6529-6532.
[52] J. Wang, J. Lu, S. Su, J. Gao, Q. Huang, L. Wang, W. Huang, X. Zuo, Biosens. Bioelectron. 2015, 65, 171-175.
[53] J. Wang, L. Wang, X. Liu, Z. Liang, S. Song, W. Li, G. Li, C. Fan, Adv. Mater. 2007, 19, 3943-3946.
[54] A. Fire, S. Q. Xu, Proc. Natl. Acad. Sci. USA 1995, 92, 4641-4645
[55] D. Liu, S. L. Daubendiek, M. A. Zillman, K. Ryan, E. T. Kool, J. Am. Chem. Soc. 1996, 118, 1587-1594.
[56] C. Larsson, J. Koch, A. Nygren, G. Janssen, A. K. Raap, U. Landegren, M. Nilsson, Nat Methods 2004, 1, 227-232
[57] P. M. Lizardi, X. Huang, Z. Zhu, P. Bray-Ward, D. C. Thomas, D. C. Ward, Nat. Genet. 1998, 19, 225-232
[58] Y. Weizmann, M. K. Beissenhirtz, Z. Cheglakov, R. Nowarski, M. Kotler, I. Willner, Angew Chem Int Ed 2006, 45, 7384-7388;
[59] S. He, L. Qu, Z. Shen, Y. Yan, M. Zeng, F. Liu, Y. Jiang, Y. Li, Anal. Chem., 2015, 87, 569-577.
[60] Pan, T.; Uhlenbeck, O. C. Nature 1992, 358, 560.
[61] Williams, K. P.; Ciafre, S.; Tocchini-Valentini, G. P. The EMBO journal 1995, 14, 4551.
[62] Santoro, S. W.; Joyce, G. F.; Sakthivel, K.; Gramatikova, S.; Barbas, C. F., 3rd Journal of the American Chemical Society 2000, 122, 2433.
[63] Cruz, R. P.; Withers, J. B.; Li, Y. Chemistry & biology 2004, 11, 57.

1. A sensor for detecting a target comprising:
a) a probe that is able to recognize the presence of the target;
b) a pH-changing enzyme conjugated to an oligonucleotide that senses the recognition of the presence of the target by the probe; and
c) a solid support linked or linkable to the probe;
wherein the presence of the target causes the sensor to be either captured to the solid support or released into solution for detection of pH changes.

2. The sensor of claim 1, wherein the pH changing enzyme is urease.

3. The sensor of claim 1, wherein the solid support is a magnetic bead, glass, plastic or paper.

4. The sensor of claim 1, wherein the target is a DNA, an RNA, a protein, a small molecule, a cell, a chemical compound or an ion.

5. The sensor of claim 1, wherein the probe is a DNA, RNA, DNAzyme, ribozyme, an aptamer, an amplified DNA product, or an aptazyme.

6. The sensor of claim 1, wherein the probe is:
a) an RNA cleaving DNAzyme having an RNA linkage that cleaves the RNA linkage in the presence of target, and wherein the oligonucleotide conjugated to the pH-changing enzyme is complementary to a portion of the RNA-containing sequence that when cleaved by the DNAzyme is no longer linked to the solid support;
b) a biotinylated primer capable of acting as a forward primer, or reverse primer, to amplify a portion of the target and wherein the oligonucleotide conjugated to the pH-changing enzyme is complementary to a portion of the reverse primer, or forward primer respectively, to amplify a portion of the target;
c) a nicked DNA having a first end and a second end and the oligonucleotide conjugated to the pH-changing enzyme is complementary to a portion of the sequence overlapping the second end and wherein the target is ATP which is required for a ligation reaction to occur;
d) a biotinylated primer and a circular DNA, wherein the primer is capable of amplifying the circular DNA by rolling circle amplification and the oligonucleotide conjugated to the pH-changing enzyme is complementary to a portion of the amplified DNA, wherein the target is the amplified DNA; or
e) a biotinylated oligonucleotide complementary to a portion of a target DNA or RNA and the oligonucleotide conjugated to the pH-changing enzyme is complementary to a different portion of the target DNA or RNA.

7. The sensor of claim 6, wherein the RNA cleaving DNAzyme of a) cleaves the RNA in the presence of uranyl ions or E. coli.

8. The sensor of claim 6, wherein the target of b) is C. difficile.

9. The sensor of claim 6, wherein the target of b) is a fungus, such as Pythium aphanidermatum.

10. The sensor of claim 6, wherein the target of e) is viral DNA.

11. A method of detecting a target in solution comprising:
a) incubating the sensor of claim 1 with the target in solution to allow the probe to recognize the target and the pH-changing enzyme-oligonucleotide conjugate to sense the recognition;
b) (i) removing the solution from the solid support after incubation if the pH-changing enzyme is releasable upon recognition of the target by the probe; or (ii) washing the solid support after incubation if the enzyme is capturable upon recognition of the target by the probe to produce a solution containing the washed solid support;
c) incubating the solution of b) i) or ii) with a substrate of the enzyme;
d) testing the pH of the solution of c)
wherein a change in pH is indicative of the presence and/or quantity of the target in the initial solution.

12. The method of claim 11, wherein the pH is tested using litmus paper or dyes or a pH meter, dye or paper.

13. The method of claim 11, wherein the pH changing enzyme is urease and substrate is urea.

14. The method of claim 11, wherein the solid support is a magnetic bead, glass, plastic or paper.

15. The method of claim 11, wherein the linking of the probe to the solid support is after the probe recognizes the target and the pH-changing enzyme-oligonucleotide conjugate senses the recognition but before b).

16. The method of claim 11, wherein the target is a DNA, an RNA, a protein, a small molecule, a cell, a chemical compound or an ion.

17. The method of claim 11, wherein the probe is a DNA, RNA, DNAzyme, ribozyme, an aptamer, an amplified DNA product, or an aptazyme.

18. The method of claim 11, wherein:
(A) the probe is an RNA cleaving DNAzyme having an RNA linkage that cleaves the RNA linkage in the presence of target, and wherein the oligonucleotide conjugated to the pH-changing enzyme is complementary to a portion of the RNA-containing sequence that when cleaved by the DNAzyme is no longer linked to the solid support and is released into solution after a); wherein in b) i) the solution is removed and wherein in c) the substrate is added to the removed solution; and wherein in d) the pH of the removed solution is tested;
(B) the probe is a biotinylated primer capable of acting as a forward primer to amplify a portion of target and the oligonucleotide conjugated to the pH-changing enzyme is complementary to a portion of the reverse primer to amplify a portion of the target, such that the amplified product is linked to the solid support and the urease is attached to the end of the amplified product after a), wherein the solid support is then washed in b) ii) and then wherein in c) substrate is added to the washed solid support in solution and wherein in d) the pH of the solid support in solution is tested;
(C) wherein the probe is a nicked DNA having a first end and a second end and wherein the oligonucleotide conjugated to the pH-changing enzyme is complementary to a portion of the sequence overlapping the second end and wherein the target is ATP which is required for a ligation reaction to occur; such that in the presence of ATP the ligation reaction occurs and the pH-changing enzyme binds to the ligated DNA, which is linked to the solid support after a), wherein the solid support is then washed in b) ii) and then wherein in c) substrate is added to the washed solid support in solution and wherein in d) the pH of the solid support in solution is tested;
(D) wherein the probe is a biotinylated primer and a circular DNA, wherein the primer is linkable to a solid support and is capable of amplifying the circular DNA by rolling circle amplification and wherein the oligonucleotide conjugated to the pH-changing enzyme is complementary to a portion of the amplified DNA, wherein the target is the amplified DNA and the pH-changing enzyme binds to the amplified DNA which is linked to the solid support after a), wherein the solid support is then washed in b) ii) and then wherein in c) substrate is added to the washed solid support in solution and wherein in d) the pH of the solid support in solution is tested; or
(E) wherein the probe is a biotinylated oligonucleotide complementary to a portion of a target DNA or RNA and the oligonucleotide conjugated to the pH-changing enzyme is complementary to a different portion of the target DNA or RNA; such that in the presence of the target, the pH-changing enzyme is captured and linked to the solid support after a), wherein the solid support is then washed in b) ii) and then wherein in c) substrate is added to the washed solid support in solution and wherein in d) the pH of the solid support in solution is tested.

19. A kit comprising the sensor of claim 1.

20. The kit of claim 19, further comprising a wash solution, litmus or other pH detecting dye or paper, or instructions for use.


SEQUENCE LISTING (30 sequences)

SEQ ID NO: 1 - DNA, 22 bp, Clostridium difficile
ctctactggcatttattttggt

SEQ ID NO: 2 - DNA, 22 bp, Clostridium difficile
catgaggaggtcatttctaatt

SEQ ID NO: 3 - DNA, 25 bp, Clostridium difficile
aatcaacgtaaagctgaagaagcta

SEQ ID NO: 4 - DNA, 63 bp, Clostridium difficile
  misc_feature (38)..(38): n is  internal triethylene glycol spacer
cttgacactattacgcacgcttcactattttttttttntaccagtatcatatcctttcttctc

SEQ ID NO: 5 - DNA, 40 bp, Artificial Sequence
  Synthetic E. coli RNA Substrate
ttttttttttttactcttcctagcnnnggttcgatcaaga

SEQ ID NO: 6 - DNA, 94 bp, Artificial Sequence
  Synthetic E. coli DNAzyme
gatgtgcgttgtcgagacctgcgaccggaacactacactgtgtggggatggatttctttacagttgtgtgttgaacgctgtgtcaaaaaaaaaa

SEQ ID NO: 7 - DNA, 26 bp, Artificial Sequence
  Synthetic E. coli Template
gacaacgcacatctcttgatcgaacc

SEQ ID NO: 8 - DNA, 30 bp, Artificial Sequence
  Synthetic E. coli Urease DNA
ttttttttttttttttgacacagcgttcaa

SEQ ID NO: 9 - DNA, 30 bp, Artificial Sequence; identical to SEQ ID NO: 8
  Synthetic E. coli Urease Reporter

SEQ ID NO: 10 - DNA, 73 bp, Artificial Sequence
  Synthetic UO2 RNA Substrate
ttttttttttttactcactatnggaagagatggacgtgtttttagggcaagtctctaatacgcacgcatcaca

SEQ ID NO: 11 - DNA, 52 bp, Artificial Sequence
  Synthetic UO2 DNAzyme
cacgtccatctctgcagtcgggtagttaaaccgaccttcagacatagtgagt

SEQ ID NO: 12 - DNA, 36 bp, Artificial Sequence
  Synthetic UO2 Urease DNA
tttttttgtgatgcgtgcgtattagagacttgccct

SEQ ID NO: 13 - DNA, 102 bp, Pythium aphanidermatum
gtagtctgccgatgtatttttcaaacccatttacctaatactgatctatactccaaaaacgaaagtttatggttttaatctataacaactttcagcagtgga

SEQ ID NO: 14 - DNA, 18 bp, Pythium aphanidermatum
gtagtctgccgatgtatt

SEQ ID NO: 15 - DNA, 51 bp, Artificial Sequence
  Synthetic primer
cgtgacctaccttacctcttgaccttgaaaaaantccactgctgaaagttg

SEQ ID NO: 16 - DNA, 33 bp, Artificial Sequence
  Synthetic PA Urease DNA
aaaaaacaaggtcaagaggtaaggtaggtcacg

SEQ ID NO: 17 - DNA, 32 bp, Artificial Sequence
  Synthetic ATP Urease DNA
ttttttagtgaagcgtgcgtaatagtgtcaag

SEQ ID NO: 18 - DNA, 88 bp, Artificial Sequence
  Synthetic ATP Full Length
ttttttttttttctatgaactgactatgacctcactaccaagaacgcttacaatgacactcccttgacactattacgcacgcttcact

SEQ ID NO: 19 - DNA, 42 bp, Artificial Sequence
  Synthetic ATP Acceptor Sequence
ttttttttttttctatgaactgactatgacctcactaccaag

SEQ ID NO: 20 - DNA, 46 bp, Artificial Sequence
  Synthetic ATP Donor Sequence
aacgcttacaatgacactcccttgacactattacgcacgcttcact

SEQ ID NO: 21 - DNA, 24 bp, Artificial Sequence
  Synthetic ATP Template
attgtaagcgttcttggtagtgag

SEQ ID NO: 22 - DNA, 30 bp, Artificial Sequence
  Synthetic ATP Blocker
cttggtagtgaggtcatagtcagttcatag

SEQ ID NO: 23 - DNA, 60 bp, Artificial Sequence
  Synthetic RCA Circular DNA
aaggagtgaagcgtgcgtaatagtgtcaaggaattcaatcaacgtaaagctgaagaagct

SEQ ID NO: 24 - DNA, 16 bp, Artificial Sequence
  Synthetic RCA Template
ctccttagcttcttca

SEQ ID NO: 25 - DNA, 32 bp, Artificial Sequence; identical to SEQ ID NO: 17
  Synthetic RCA Urease DNA

SEQ ID NO: 26 - DNA, 16 bp, Artificial Sequence
  Synthetic RCA Primer
ctccttagcttcttca

SEQ ID NO: 27 - DNA, 60 bp, Artificial Sequence
  Synthetic RCA Primer
tcttcagctttacgttgattgaattccttgacactattacgcacgcttcactccttagct

SEQ ID NO: 28 - DNA, 20 bp, Hepatitis C virus
agtccaccgtgtcgtctgct

SEQ ID NO: 29 - DNA, 22 bp, Artificial Sequence
  Synthetic HCV Capture
acggtggacttttttttttttt

SEQ ID NO: 30 - DNA, 19 bp, Artificial Sequence
  Synthetic HCV Urease DNA
tttttttttagcagacgac